(19) United States
(12) Patent Application Publication
(10) Pub. No.: US 2016/0160294 A1
(43) Pub. Date: Jun. 9, 2016

(54) METHODS AND MATERIALS FOR PREDICTING RESPONSE TO NIRAPARIB

(71) Applicants: Tesaro, Waltham, MA (US); Myriad Genetics, Inc., Salt Lake City, UT (US)

(72) Inventors: Keith Wilcoxen, Waltham, MA (US); Alexander Gutin, Salt Lake City, UT (US); Kirsten Timms, Salt Lake City, UT (US); Victor Abkevich, Salt Lake City, UT (US); Yan Wang, Waltham, MA (US); Jerry Lanchbury, Salt Lake City, UT (US)

(21) Appl. No.: 14/962,588

(22) Filed: Dec. 8, 2015

Related U.S. Application Data
(60) Provisional application No. 62/089,077, filed on Dec. 8, 2014.
(60) Provisional application No. 62/166,387, filed on May 26, 2015.

(51) Int. Cl. C12Q 1/68; A61K 31/454

(52) U.S. Cl. CPC C12Q 1/6886; A61K 31/454; C12Q 2600/106; C12Q 2600/156

(57) ABSTRACT

This document provides methods and materials involved in assessing samples (e.g., cancer cells) for the presence of homologous recombination deficiency (HRD) or an HRD signature to predict response to niraparib. For example, methods and materials for determining whether or not a cell (e.g., a cancer cell) contains an HRD signature to predict response to niraparib are provided.

This application is claims priority to U.S. Provisional Patent Application Ser. No. 62/089,077 filed Dec. 8, 2014 and U.S. Provisional Patent Application Ser. No. 62/166,387 filed May 26, 2015, the entire contents of which are hereby incorporated by reference.

### BACKGROUND

Cancer is a serious public health problem, with 562,340 people in the United States of America dying of cancer in 2009 alone. American Cancer Society, Cancer Facts & Figures 2009 (available at American Cancer Society website). One of the primary challenges in cancer treatment is discovering relevant, clinically useful characteristics of a patient's own cancer and then, based on these characteristics, administering a treatment plan best suited to the patient's cancer. Triple negative breast cancer (TNBC) has a poor prognosis and currently lacks effective treatment. TNBCs are highly proliferative, genomically unstable and share molecular characteristics with that of BRCA1/2 mutation driven breast cancer.

Poly(ADP-ribose) polymerase-1 (PARP) is a key DNA repair enzyme that mediates single strand break (SSB) repair through the base excision repair (BER) pathway. PARP inhibitors have been demonstrated to selectively kill tumor cells that harbor BRCA1 and BRCA2 mutations. In addition, pre-clinical and preliminary clinical data suggest that PARP inhibitors are selectively cytotoxic for tumors with homologous recombination repair deficiency caused by dysfunction of genes other than BRCA1 or BRCA2. Niraparib is a potent, orally active PARP inhibitor that is being evaluated in Phase 3 clinical studies for ovarian cancer and BRCA related breast cancer. Previously, it was demonstrated that a subset of basal breast cancer (BBC) patient-derived xenograft (PDX) models responded robustly to single agent niraparib treatment (Wong et al., Clin. Cancer Res. 18; 3846-55 (2012)). However, there is a need for better molecular diagnostic tools to predict which cancer patients will respond best to PARP inhibitors.

### SUMMARY

In one embodiment, an in vitro method of predicting a triple negative breast cancer patient response to a cancer treatment regimen comprising niraparib is provided. The method comprises (1) determining in a patient sample the number of Indicator CA Regions comprising at least two types chosen from Indicator LOH Regions, Indicator TAI Regions, or Indicator LST Regions in at least two pairs of human chromosomes of a breast cancer cell; and (2) diagnosing a patient in whose sample said number of Indicator LOH Regions, Indicator TAI Regions, and/or Indicator LST Regions is greater than a reference number as having an increased likelihood of responding to said cancer treatment regimen. In an embodiment, at least two pair of human chromosomes is representative of the entire genome. In an embodiment, said Indicator CA Regions are determined in at least two, three, four, five, six, seven, eight, nine, ten, 11, 12, 13, 14, 15, 16, 17, 18, 19, 20 or 21 pairs of human chromosomes. In an embodiment, the Indicator CA Regions analyzed in said patient sample comprise Indicator LOH Regions, Indicator TAI Regions, and Indicator LST Regions. In an embodiment, a Combined CA Region Score is calculated from the number of Indicator CA Regions and in step (2) a patient in whose sample said Combined CA Region Score is greater than a reference Combined CA Region Score is diagnosed as having an increased likelihood of responding to said cancer treatment regimen. In an embodiment, the reference number is 5, 6, 7, 8, 9, 10, 11, 12, 13, 14, 15, 16, 18, 19, 20, 22, 24, 26, 28, 30, 32, 34, 36, 38, 40, 42, 44, 46, 48, 50 or greater. In an embodiment, the reference number is 42. In an embodiment, the reference Combined CA Region Scores is 5, 6, 7, 8, 9, 10, 11, 12, 13, 14, 15, 16, 18, 19, 20, 22, 24, 26, 28, 30, 32, 34, 36, 38, 40, 42, 44, 46, 48, 50 or greater. In an embodiment, the reference Combined CA Region Score is 42. In an embodiment, the method further comprises administering said cancer treatment regimen to said patient diagnosed as having an increased likelihood of responding to said cancer treatment regimen. In an embodiment, the step of determining comprises hybridizing nucleic acids from the sample with probes.

In another embodiment, an in vitro method of predicting patient response to a cancer treatment regimen comprising niraparib is provided. The method comprises (1) determining in a patient sample the number of Indicator CA Regions comprising at least two types chosen from Indicator LOH Regions, Indicator TAI Regions, or Indicator LST Regions in at least two pairs of human chromosomes of a breast cancer cell; (2) providing a test value derived from the number of said Indicator CA Regions; (3) comparing said test value to one or more reference values derived from the number of said Indicator CA Regions in a reference population; and (4) diagnosing a patient in whose sample said test value is greater than said one or more reference numbers as having an increased likelihood of responding to said cancer treatment regimen. In an embodiment, at least two pair of human chromosomes is representative of the entire genome. In an embodiment, said Indicator CA Regions are determined in at least two, three, four, five, six, seven, eight, nine, ten, 11, 12, 13, 14, 15, 16, 17, 18, 19, 20 or 21 pairs of human chromosomes. In an embodiment, the Indicator CA Regions analyzed in said patient sample comprise Indicator LOH Regions, Indicator TAI Regions, and Indicator LST Regions. In an embodiment, a Combined CA Region Score is calculated from the number of Indicator CA Regions and in step (2) a patient in whose sample said Combined CA Region Score is greater than a reference Combined CA Region Score is diagnosed as having an increased likelihood of responding to said cancer treatment regimen. In an embodiment, the reference number is 5, 6, 7, 8, 9, 10, 11, 12, 13, 14, 15, 16, 18, 19, 20, 22, 24, 26, 28, 30, 32, 34, 36, 38, 40, 42, 44, 46, 48, 50 or greater. In an embodiment, the reference number is 42. In an embodiment, the reference Combined CA Region Scores is 5, 6, 7, 8, 9, 10, 11, 12, 13, 14, 15, 16, 18, 19, 20, 22, 24, 26, 28, 30, 32, 34, 36, 38, 40, 42, 44, 46, 48, 50 or greater. In an embodiment, the reference Combined CA Region Score is 42. In an embodiment, the method further comprises diagnosing a patient in whose sample said test value is not greater than said one or more reference numbers as not having an increased likelihood of responding to said cancer treatment regimen and either (5)(a) recommending, prescribing, initiating or continuing a treatment regimen comprising niraparib in said patient diagnosed as having an increased likelihood of responding to said cancer treatment regimen; or (5)(b) recommending, prescribing, initiating or continuing a treatment regimen not comprising niraparib in said patient diagnosed as not having an increased likelihood of responding to said cancer treatment regimen. In an embodiment, the method comprises diagnosing a patient in whose sample said test value is at least 2-, 3-, 4-, 5-, 6-, 7-, 8-, 9-, or 10-fold greater, at least 1, 2, 3, 4, 5, 6, 7, 8, 9, or 10 standard deviations greater, or at least 5%, 10%, 15%, 20%, 25%, 30%, 35%, 40%, 45%, 50%, 55%, 60%, 65%, 70%, 75%, 80%, 85%, 90%, 95% greater than said one or more reference numbers as having an increased likelihood of responding to said cancer treatment regimen. In an embodiment, the method comprises hybridizing nucleic acids from the sample with probes.

In another embodiment, a method of treating triple negative breast cancer patients is provided. The method comprises (1) determining in a patient sample the number of Indicator CA Regions comprising Indicator LOH Regions, Indicator TAI Regions, and Indicator LST Regions in at least two pairs of human chromosomes of a breast cancer cell; (2) providing a test value derived from the number of said Indicator CA Regions; (3) comparing said test value to one or more reference values derived from the number of said Indicator CA Regions in a reference population; and either (4)(a) recommending, prescribing, initiating or continuing a treatment regimen comprising niraparib in a patient in whose sample the test value is greater than at least one said reference value; or (4)(b) recommending, prescribing, initiating or continuing a treatment regimen comprising niraparib in a patient in whose sample the test value is not greater than at least one said reference value. In an embodiment, said at least two pair of human chromosomes is representative of the entire genome. In an embodiment, said Indicator CA Regions are determined in at least two, three, four, five, six, seven, eight, nine, ten, 11, 12, 13, 14, 15, 16, 17, 18, 19, 20 or 21 pairs of human chromosomes. In an embodiment, the Indicator CA Regions analyzed in said patient sample comprise Indicator LOH Regions, Indicator TAI Regions, and Indicator LST Regions. In an embodiment, a Combined CA Region Score is calculated from the number of Indicator CA Regions and in step (2) a patient in whose sample said Combined CA Region Score is greater than a reference Combined CA Region Score is diagnosed as having an increased likelihood of responding to said cancer treatment regimen. In an embodiment, the reference number is 5, 6, 7, 8, 9, 10, 11, 12, 13, 14, 15, 16, 18, 19, 20, 22, 24, 26, 28, 30, 32, 34, 36, 38, 40, 42, 44, 46, 48, 50 or greater. In an embodiment, the reference number is 42. In an embodiment, the reference Combined CA Region Scores is 5, 6, 7, 8, 9, 10, 11, 12, 13, 14, 15, 16, 18, 19, 20, 22, 24, 26, 28, 30, 32, 34, 36, 38, 40, 42, 44, 46, 48, 50 or greater. In an embodiment, the reference Combined CA Region Score is 42. In an embodiment, the method comprises diagnosing a patient in whose sample said test value is at least 2-, 3-, 4-, 5-, 6-, 7-, 8-, 9-, or 10-fold greater, at least 1, 2, 3, 4, 5, 6, 7, 8, 9, or 10 standard deviations greater, or at least 5%, 10%, 15%, 20%, 25%, 30%, 35%, 40%, 45%, 50%, 55%, 60%, 65%, 70%, 75%, 80%, 85%, 90%, 95% greater than said one or more reference numbers as having an increased likelihood of responding to said cancer treatment regimen. In an embodiment, the determining comprises hybridizing nucleic acids from the sample with probes.

In another embodiment, a method of analyzing a tumor sample is provided. The method comprises the steps of (1) providing a tumor sample from a subject suffering from cancer, whose potential response to a cancer treatment regimen comprising niraparib is to be determined, wherein the tumor sample comprises nucleic acid; (2) determining a value for Indicator CA Regions in the nucleic acid, which value incorporates numbers of at least two types of Indicator Regions selected from the group consisting of Indicator LOH Regions, Indicator TAI Regions, Indicator LST Regions, and combinations thereof, for at least two pairs of human chromosomes from cells in the tumor sample; and (3) comparing the determined value with that of a corresponding reference value from a comparable tumor sample or samples from tumor(s) with known responsiveness to niraparib. In an embodiment, at least two pair of human chromosomes is representative of the entire genome. In an embodiment, said Indicator CA Regions are determined in at least two, three, four, five, six, seven, eight, nine, ten, 11, 12, 13, 14, 15, 16, 17, 18, 19, 20 or 21 pairs of human chromosomes. In an embodiment, the Indicator CA Regions analyzed in said patient sample comprise Indicator LOH Regions, Indicator TAI Regions, and Indicator LST Regions. In an embodiment, a Combined CA Region Score is calculated from the number of Indicator CA Regions and in step (2) a patient in whose sample said Combined CA Region Score is greater than a reference Combined CA Region Score is diagnosed as having an increased likelihood of responding to said cancer treatment regimen. In an embodiment, the reference number is 5, 6, 7, 8, 9, 10, 11, 12, 13, 14, 15, 16, 18, 19, 20, 22, 24, 26, 28, 30, 32, 34, 36, 38, 40, 42, 44, 46, 48, 50 or greater. In an embodiment, the reference number is 42. In an embodiment, the reference Combined CA Region Scores is 5, 6, 7, 8, 9, 10, 11, 12, 13, 14, 15, 16, 18, 19, 20, 22, 24, 26, 28, 30, 32, 34, 36, 38, 40, 42, 44, 46, 48, 50 or greater. In an embodiment, the reference Combined CA Region Score is 42. In an embodiment, the method further comprises administering said cancer treatment regimen to said patient diagnosed as having an increased likelihood of responding to said cancer treatment regimen. In an embodiment, the step of determining comprises hybridizing nucleic acids from the sample with probes.

The details of one or more embodiments of the invention are set forth in the description and accompanying drawings below. The materials, methods, and examples are illustrative only and not intended to be limiting. Other features, objects, and advantages of the invention will be apparent from the description and drawings, and from the claims.

### DESCRIPTION OF THE DRAWINGS

FIG. 1 shows graphs plotting allele dosages of breast cancer cells from a fresh frozen sample from a breast cancer patient along a chromosome as determined using a SNP array (above) and high-throughput sequencing (below).

FIG. 2 shows graphs plotting allele dosages of breast cancer cells from an FFPE sample from a breast cancer patient along a chromosome as determined using a SNP array (above) and high-throughput sequencing (below).

FIG. 3 is a flow chart of an example process for assessing the genome of a cell (e.g., a cancer cell) for an HRD signature.

FIG. 4 is a diagram of an example of a computer device and a mobile computer device that can be used to implement the techniques described herein.

FIG. 5 is a graph plotting the number of LOH regions longer than 15 Mb and shorter than the entire chromosome for ovarian cancer cell samples with somatic BRCA mutations, with germline BRCA mutations, with low BRCA1 expression, or with intact BRCA (BRCA normal). The size of the circles is proportional to the number of samples with such number of LOH regions.

FIG. 6A illustrates the distribution of HRD scores in the training set of tumors. FIG. 6A is the breast tumor sample data.

FIG. 6B illustrates the distribution of HRD scores in the training set of tumors. FIG. 6B is the combined breast and ovarian tumor sample data.

FIG. 7 demonstrates that the niraparib sensitive models are HRD-positive.

FIG. 8 illustrates how niraparib treatment reduced tumor volume.

FIG. 9 illustrates that all 6 niraparib sensitive TNBC models (6 right-most bars) had an HRD score higher than 42.

FIG. 10 illustrates the ability of HRD score as described herein to separate sensitive from insensitive ovarian cancer PDX models.

FIG. 11 illustrates example growth curves of ovarian cancer PDX models found to be sensitive to niraparib.

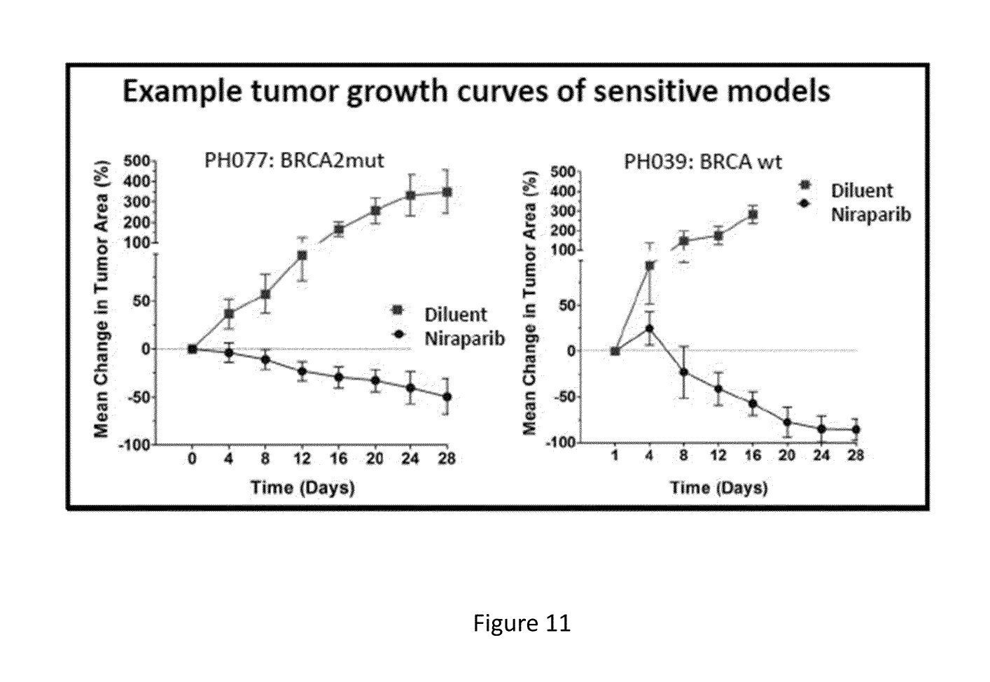

### DETAILED DESCRIPTION

In general, one aspect of this invention features a method for predicting response to (or identifying patients appropriate for, or optimizing therapy with, etc.) treatment comprising niraparib, the method comprising, or consisting essentially of, (a) detecting, in a sample or DNA derived therefrom, CA Regions in at least one pair of human chromosomes or DNA derived therefrom; (b) determining the number, size (e.g., length), and/or character of said CA Regions; and (c) diagnosing (identifying, etc.) a patient in whose sample CA Regions of a certain character are detected as having a high likelihood of response to treatment comprising niraparib.

As used herein, “chromosomal aberration” or “CA” means a somatic change in a cell's chromosomal DNA that falls into at least one of three overlapping categories: LOH, TAI, or LST. Polymorphic loci within the human genome (e.g., single nucleotide polymorphisms (SNPs)) are generally heterozygous within an individual's germline since that individual typically receives one copy from the biological father and one copy from the biological mother. Somatically, however, this heterozygosity can change (via mutation) to homozygosity. This change from heterozygosity to homozygosity is called loss of heterozygosity (LOH). LOH may result from several mechanisms. For example, in some cases, a locus of one chromosome can be deleted in a somatic cell. The locus that remains present on the other chromosome (the other non-sex chromosome for males) is an LOH locus as there is only one copy (instead of two copies) of that locus present within the genome of the affected cells. This type of LOH event results in a copy number reduction. In other cases, a locus of one chromosome (e.g., one non-sex chromosome for males) in a somatic cell can be replaced with a copy of that locus from the other chromosome, thereby eliminating any heterozygosity that may have been present within the replaced locus. In such cases, the locus that remains present on each chromosome is an LOH locus and can be referred to as a copy neutral LOH locus. LOH and its use in determining HRD is described in detail in International Application no. PCT/US2011/040953 (published as WO/2011/160063), the entire contents of which are incorporated herein by reference.

A broader class of chromosomal aberration, which encompasses LOH, is allelic imbalance. Allelic imbalance occurs when the relative copy number (i.e., copy proportion) at a particular locus in somatic cells differs from the germline. For example, if the germline has one copy of allele A and one copy of allele B at a particular locus, and a somatic cell has two copies of A and one copy of B, there is allelic imbalance at the locus because the copy proportion of the somatic cell (2:1) differs from the germline (1:1). LOH is an example of allelic imbalance since the somatic cell has a copy proportion (1:0 or 2:0) that differs from the germline (1:1). But allelic imbalance encompasses more types of chromosomal aberration, e.g., 2:1 germline going to 1:1 somatic; 1:0 germline going to 1:1 somatic; 1:1 germline going to 2:1 somatic, etc. Analysis of regions of allelic imbalance encompassing the telomeres of chromosomes is particularly useful in the invention. Thus, a “telomeric allelic imbalance region” or “TAI Region” is defined as a region with allelic imbalance that (a) extends to one of the subtelomeres and (b) does not cross the centromere. TAI and its use in determining HRD is described in detail in International Application no. PCT/US2011/048427 (published as WO/2012/027224), the entire contents of which are incorporated herein by reference.

A class of chromosomal aberrations that is broader still, which encompasses LOH and TAI, is referred to herein as large scale transition (“LST”). LST refers to any somatic copy number transition (i.e., breakpoint) along the length of a chromosome where it is between two regions of at least some minimum length (e.g., at least 3, 4, 5, 6, 7, 8 9, 10, 11 12, 13, 14, 15, 16, 17, 18, 19 or 20 or more megabases) after filtering out regions shorter than some maximum length (e.g., 0.1, 0.2, 0.3, 0.4, 0.5, 0.6, 0.7, 0.8, 0.9, 1, 1.5, 2, 2.5, 3, 3.5, 4 or more megabases). For example, if after filtering out regions shorter than 3 megabases the somatic cell has a copy number of 1:1 for, e.g., at least 10 megabases and then a breakpoint transition to a region of, e.g., at least 10 megabases with copy number 2:2, this is an LST. An alternative way of defining the same phenomenon is as an LST Region, which is genomic region with stable copy number across at least some minimum length (e.g., at least 3, 4, 5, 6, 7, 8, 9, 10, 11 12, 13, 14, 15, 16, 17, 18, 19 or 20 megabases) bounded by breakpoints (i.e., transitions) where the copy number changes for another region also at least this minimum length. For example, if after filtering out regions shorter than 3 megabases the somatic cell has a region of at least 10 megabases with copy number of 1:1 bounded on one side by a breakpoint transition to a region of, e.g., at least 10 megabases with copy number 2:2, and bounded on the other side by a breakpoint transition to a region of, e.g., at least 10 megabases with copy number 1:2, then this is two LSTs. Notice that this is broader than allelic imbalance because such a copy number change would not be considered allelic imbalance (because the copy proportions 1:1 and 2:2 are the same, i.e., there has been no change in copy proportion). LST and its use in determining HRD is described in detail in Popova et al., Ploidy and large-scale genomic instability consistently identify basal-like breast carcinomas with BRCA1/2 inactivation, CANCER RES. (2012) 72:5454-5462.

Different cutoffs for LST score may be used for “near-diploid” and “near-tetraploid” tumors to separate BRCA1/2 intact and deficient samples. LST score sometimes increases with ploidy both within intact and deficient samples. As an alternative to using ploidy-specific cutoffs, some embodiments may employ a modified LST score adjusting it by ploidy: LSTm=LST−kP, where P is ploidy and k is a constant. Based on multivariate logistic regression analysis with deficiency as an outcome and LST and P as predictors, k=15.5 provided the best separation between intact and deficient samples (though one skilled in the art can envisage other values for k).

Chromosomal aberrations can extend across numerous loci to define a region of chromosomal aberration, referred to herein as a “CA Region.” Such CA Regions can be any length (e.g., from a length less than about 1.5 Mb up to a length equal to the entire length of the chromosome). An abundance of large CA Regions (“Indicator CA Regions”) indicate a deficiency in the homology-dependent repair (HDR) mechanism of a cell. The definition of a region of CA, and thus what constitutes an “Indicator” region, for each type of CA (e.g., LOH, TAI, LST) depends on the particular character of the CA. For example, an “LOH Region” means at least some minimum number of consecutive loci exhibiting LOH or some minimum stretch of genomic DNA having consecutive loci exhibiting LOH. A “TAI Region,” on the other hand, means at least some minimum number of consecutive loci exhibiting allelic imbalance extending from the telomere into the rest of the chromosome (or some minimum stretch of genomic DNA extending from the telomere into the rest of the chromosome having consecutive loci exhibiting allelic imbalance). LST is already defined in terms of a region of genomic DNA of at least some minimum size, so “LST” and “LST Region” are used interchangeably in this document to refer to a minimum number of consecutive loci (or some minimum stretch of genomic DNA) having the same copy number bounded by a breakpoint or transition from that copy number to a different one.

In some embodiments a CA Region (whether an LOH Region, TAI region, or LST Region) is an Indicator CA Region (whether an Indicator LOH Region, Indicator TAI region, or Indicator LST Region) if it is at least 3, 4, 5, 6, 7, 8, 9, 10, 11, 12, 13, 14, 15, 16, 17, 18, 19, 20, 25, 30, 35, 40, 45, 50, 60, 70, 80, 90, or 100 megabases or more in length. In some embodiments, Indicator LOH Regions are LOH Regions that are longer than about 1.5, 5, 12, 13, 14, 15, 16, 17 or more (preferably 14, 15, 16 or more, more preferably 15 or more) megabases but shorter than the entire length of the respective chromosome within which the LOH Region is located. Alternatively or additionally, the total combined length of such Indicator LOH Regions may be determined. In some embodiments, Indicator TAI Regions are TAI Regions with allelic imbalance that (a) extend to one of the subtelomeres, (b) do not cross the centromere and (c) are longer than 1.5, 5, 12, 13, 14, 15, 16, 17 or more (preferably 10, 11, 12 or more, more preferably 11 or more) megabases. Alternatively or additionally, the total combined length of such Indicator TAI Regions may be determined. Because the concept of LST already involves regions of some minimum size (such minimum size being determined based on its ability to differentiate HRD from HDR intact samples), Indicator LST Regions as used herein are the same as LST Regions. Furthermore, an LST Region Score can be either derived from the number of regions showing LST as described above or the number of LST breakpoints. In some embodiments the minimum length of the region of stable copy number bounding the LST breakpoint is at least 3, 4, 5, 6, 7, 8, 9, 10, 11 12, 13, 14, 15, 16, 17, 18, 19 or 20 megabases (preferably 8, 9, 10, 11 or more megabases, more preferably 10 megabases) and the maximum region remaining unfiltered is less than 0.1, 0.2, 0.3, 0.4, 0.5, 0.6, 0.7, 0.8, 0.9, 1, 1.5, 2, 2.5, 3, 3.5, 4 or fewer megabases (preferably 2, 2.5, 3, 3.5, or 4 or fewer megabases, more preferably fewer than 3 megabases).

As used herein, a sample has an “HRD signature” if such sample has a number of Indicator CA Regions (as described herein) or a CA Region Score (as described herein) exceeding a reference as described herein, wherein a number or score exceeding such reference indicates homologous recombination deficiency.

Thus, some aspects of the invention include detecting and quantifying Indicator CA Regions in a sample to determine whether cells in the sample (or cells from which DNA in the sample are derived) have an HRD signature (the presence of which can then be used to diagnose a patient as having a high likelihood of response to treatment comprising niraparib. Often this comprises comparing the number of Indicator CA Regions (or a test value or score derived or calculated therefrom and corresponding to such number) to a reference, or index number (or score). In some embodiments, as described herein, a test value or score is considered to be “higher than” or “elevated relative to” an appropriate reference or index number or score when it reflects a larger number of Indicator CA Regions and/or less active HRD in the cells from which the test sample originates relative to those from which the reference sample originates.

The present invention comprises using a combined analysis of two or more types of CA Regions (including two or more types of Indicator CA Regions) to assess (e.g., detect, diagnose) HRD in a sample. Thus, in some aspects the invention comprises (1) determining the total number (or combined length) of Indicator LOH Regions in a sample; (2) determining the total number (or combined length) of Indicator TAI Regions in the sample; (3) determining the presence or absence of (e.g., detecting, diagnosing) HRD in the sample based at least in part on the determinations made in (1) and (2); and (4) and diagnosing, predicting, etc. niraparib response as described herein. In other aspects the invention comprises (1) determining the total number (or combined length) of Indicator LOH Regions in the sample; (2) determining the total number (or combined length) of Indicator LST Regions in the sample; (3) determining the presence or absence of (e.g., detecting, diagnosing) HRD in the sample based at least in part on the determinations made in (1) and (2); and (4) and diagnosing, predicting, etc. niraparib response as described herein. In another aspect the invention comprises (1) determining the total number (or combined length) of Indicator TAI Regions in the sample; (2) determining the total number (or combined length) of Indicator LST Regions in the sample; (3) determining the presence or absence of (e.g., detecting, diagnosing) HRD in the sample based at least in part on the determinations made in (1) and (2); and (4) and diagnosing, predicting, etc. niraparib response as described herein. In another aspect the invention comprises (1) determining the total number (or combined length) of Indicator LOH Regions in the sample; (2) determining the total number of Indicator TAI Regions in the sample; (3) determining the total number (or combined length) of Indicator LST Regions in the sample; (4) determining the presence or absence of (e.g., detecting, diagnosing) HRD in the sample based at least in part on the determinations made in (1), (2) and (3); and (5) and diagnosing, predicting, etc. niraparib response as described herein.

Some aspects of the present invention comprise using a combined analysis of the averages of three different CA Regions to assess (e.g., detect, diagnose) HRD in a sample. Thus, in some aspects the invention comprises (1) determining the total number of LOH Regions of a certain size or character (e.g., “Indicator LOH Regions”, as defined herein) in the sample; (2) determining the total number of TAI Regions of a certain size or character (e.g., “Indicator TAI Regions”, as defined herein) in the sample; (3) determining the total number of LST Regions of a certain size or character (e.g., “Indicator LST Regions”, as defined herein) in the sample; (4) calculating the average (e.g., arithmetic mean) of the determinations made in (1), (2), and (3); (5) assessing HRD in the sample based at least in part on the calculated average (e.g., arithmetic mean) made in (4); and (6) and diagnosing, predicting, etc. niraparib response as described herein.

As used herein, “CA Region Score” means a test value or score derived or calculated from (e.g., representing or corresponding to) Indicator CA Regions detected in a sample (e.g., a score or test value derived or calculated from the number of Indicator CA Regions detected in a sample). Analogously, as used herein, “LOH Region Score” is a subset of CA Region Scores and means a test value or score derived or calculated from (e.g., representing or corresponding to) Indicator LOH Regions detected in a sample (e.g., a score or test value derived or calculated from the number of Indicator LOH Regions detected in a sample), and so on for TAI Region Score and LST Region Score. Such a score may in some embodiments be simply the number of Indicator CA Regions detected in a sample. In some embodiments the score is more complicated, factoring in the lengths of each Indicator CA Region or a subset of Indicator CA Regions detected.

As discussed above, the invention will generally involve combining the analysis of two or more types of CA Region Scores (which may include the number of such regions) to assess HRD (and then diagnose, predict, etc. niraparib response as described herein). Thus, in some aspects of the invention assessing (e.g., detecting, diagnosing) HRD in a sample comprises (1) determining an LOH Region Score for the sample; (2) determining a TAI Region Score for the sample; (3)(a) detecting (or diagnosing) HRD in the sample based at least in part on either the LOH Region Score exceeding a reference or the TAI Region Score exceeding a reference; or optionally (3)(b) detecting (or diagnosing) an absence of HRD in the sample based at least in part on both the LOH Region Score not exceeding a reference and the TAI Region Score not exceeding a reference. In other aspects of the invention assessing (e.g., detecting, diagnosing) HRD in a sample comprises (1) determining an LOH Region Score for the sample; (2) determining an LST Region Score for the sample; and (3)(a) detecting (or diagnosing) HRD in the sample based at least in part on either the LOH Region exceeding a reference or the LST Region Score exceeding a reference; or optionally (3)(b) detecting (or diagnosing) an absence of HRD in the sample based at least in part on both the LOH Region Score not exceeding a reference and the LST Region Score not exceeding a reference. In other aspects of the invention assessing (e.g., detecting, diagnosing) HRD in a sample comprises (1) determining a TAI Region Score for the sample; (2) determining an LST Region Score for the sample; and (3)(a) detecting (or diagnosing) HRD in the sample based at least in part on either the TAI Region Score exceeding a reference or the LST Region Score exceeding a reference; or optionally (3)(b) detecting (or diagnosing) an absence of HRD in the sample based at least in part on both the TAI Region Score not exceeding a reference and the LST Region Score not exceeding a reference. In other aspects of the invention assessing (e.g., detecting, diagnosing) HRD in a sample comprises (1) determining an LOH Region Score for the sample; (2) determining a TAI Region Score for the sample; (3) determining an LST Region Score for the sample; and (4)(a) detecting (or diagnosing) HRD in the sample based at least in part on either the LOH Region Score exceeding reference, the TAI Region Score exceeding a reference or the LST Region Score exceeding a reference; or optionally (4)(b) detecting (or diagnosing) an absence of HRD in the sample based at least in part on the LOH Region Score not exceeding a reference, the TAI Region Score not exceeding a reference and the LST Region Score not exceeding a reference.

In some embodiments the CA Region Score is a combination of scores derived or calculated from (e.g., representing or corresponding to) two or more of (1) the detected LOH Regions (“LOH Region Score”, as defined herein), (2) the detected TAI Regions (“TAI Region Score”, as defined herein), and/or (3) the detected LST Regions (“LST Region Score”, as defined herein). In some embodiments the LOH Region Score and TAI Region Score are combined as follows to yield a CA Region Score:

CA Region Score=A*(LOH Region Score)+B*(TAI Region Score)

In some embodiments the LOH Region Score and TAI Region Score are combined as follows to yield a CA Region Score:

CA Region Score=0.32*(LOH Region Score)+0.68*(TAI Region Score)

OR

CA Region Score=0.34*(LOH Region Score)+0.66*(TAI Region Score)

In some embodiments the LOH Region Score and LST Region Score are combined as follows to yield a CA Region Score:

CA Region Score=A*(LOH Region Score)+B*(LST Region Score)

In some embodiments an LOH Region Score for a sample and an LST Region Score for a sample are combined to yield a CA Region Score as follows:

CA Region Score=0.85*(LOH Region Score)+0.15*(LST Region Score)

In some embodiments the TAI Region Score and LST Region Score are combined as follows to yield a CA Region Score:

CA Region Score=A*(TAI Region Score)+B*(LST Region Score)

In some embodiments the LOH Region Score, TAI Region Score and LST Region Score are combined as follows to yield a CA Region Score:

CA Region Score=A*(LOH Region Score)+B*(TAI Region Score)+C*(LST Region Score)

In some embodiments the LOH Region Score, TAI Region Score and LST Region Score are combined as follows to yield a CA Region Score:

CA Region Score=0.21*(LOH Region Score)+0.67*(TAI Region Score)+0.12*(LST Region Score)

OR

CA Region Score=[0.24]*(LOH Region Score)+[0.65]*(TAI Region Score)+[0.11]*(LST Region Score)

OR

CA Region Score=[0.11]*(LOH Region Score)+[0.25]*(TAI Region Score)+[0.12]*(LST Region Score)

In some embodiments the CA Region Score is a combination of scores derived or calculated from (e.g., representing or corresponding to) the average (e.g., arithmetic mean) of (1) the detected LOH Regions (“LOH Region Score”, as defined herein), (2) the detected TAI Regions (“TAI Region Score”, as defined herein), and/or (3) the detected LST Regions (“LST Region Score”, as defined herein) to yield a CA Region Score calculated from one of the following formulae:

$$CA Region Score = \frac{\begin{matrix}{A}^{*} ( LOH Region Score ) + \\ {B}^{*} ( TAI Region Score ) + {C}^{*} ( LST Region Score )\end{matrix}}{( 3 )}$$

$$CA Region Score = \frac{{A}^{*} ( LOH Region Score ) + {B}^{*} ( TAI Region Score )}{2}$$

$$CA Region Score = \frac{{A}^{*} ( LOH Region Score ) + {C}^{*} ( LST Region Score )}{2}$$

$$CA Region Score = \frac{{B}^{*} ( TAI Region Score ) + {C}^{*} ( LST Region Score )}{2}$$

In some embodiments, including some specifically illustrated herein, one or more of these coefficients (i.e., A, B, or C, or any combination thereof) is 1 and in some embodiments all three coefficients (i.e., A, B, and C) are 1. Thus, in some embodiments the CA Region Score=(LOH Regions Score)+(TAI Region Score)+(LST Region Score), wherein the LOH Region Score is the number of Indicator LOH Regions (or the total length of LOH), the TAI Region Score is the number of Indicator TAI Regions (or the total length of TAI), and the LST Region Score is the number of Indicator LST Regions (or the total length of LST).

In some cases a formula may not have all of the specified coefficients (and thus not incorporate the corresponding variable(s)). For example, the embodiment mentioned immediately previously may be applied to formula (2) where A in formula (2) is 0.95 and B in formula (2) is 0.61. C and D would not be applicable as these coefficients and their corresponding variables are not found in formula (2) (though the clinical variables are incorporated into the clinical score found in formula (2)). In some embodiments A is between 0.9 and 1, 0.9 and 0.99, 0.9 and 0.95, 0.85 and 0.95, 0.86 and 0.94, 0.87 and 0.93, 0.88 and 0.92, 0.89 and 0.91, 0.85 and 0.9, 0.8 and 0.95, 0.8 and 0.9, 0.8 and 0.85, 0.75 and 0.99, 0.75 and 0.95, 0.75 and 0.9, 0.75 and 0.85, or between 0.75 and 0.8. In some embodiments B is between 0.40 and 1, 0.45 and 0.99, 0.45 and 0.95, 0.55 and 0.8, 0.55 and 0.7, 0.55 and 0.65, 0.59 and 0.63, or between 0.6 and 0.62. In some embodiments C is, where applicable, between 0.9 and 1, 0.9 and 0.99, 0.9 and 0.95, 0.85 and 0.95, 0.86 and 0.94, 0.87 and 0.93, 0.88 and 0.92, 0.89 and 0.91, 0.85 and 0.9, 0.8 and 0.95, 0.8 and 0.9, 0.8 and 0.85, 0.75 and 0.99, 0.75 and 0.95, 0.75 and 0.9, 0.75 and 0.85, or between 0.75 and 0.8. In some embodiments D is, where applicable, between 0.9 and 1, 0.9 and 0.99, 0.9 and 0.95, 0.85 and 0.95, 0.86 and 0.94, 0.87 and 0.93, 0.88 and 0.92, 0.89 and 0.91, 0.85 and 0.9, 0.8 and 0.95, 0.8 and 0.9, 0.8 and 0.85, 0.75 and 0.99, 0.75 and 0.95, 0.75 and 0.9, 0.75 and 0.85, or between 0.75 and 0.8.

In some embodiments A is between 0.1 and 0.2, 0.3, 0.4, 0.5, 0.6, 0.7, 0.8, 0.9, 1, 1.5, 2, 2.5, 3, 3.5, 4, 4.5, 5, 6, 7, 8, 9, 10, 11, 12, 13, 14, 15, or 20; or between 0.2 and 0.3, 0.4, 0.5, 0.6, 0.7, 0.8, 0.9, 1, 1.5, 2, 2.5, 3, 3.5, 4, 4.5, 5, 6, 7, 8, 9, 10, 11, 12, 13, 14, 15, or 20; or between 0.3 and 0.4, 0.5, 0.6, 0.7, 0.8, 0.9, 1, 1.5, 2, 2.5, 3, 3.5, 4, 4.5, 5, 6, 7, 8, 9, 10, 11, 12, 13, 14, 15, or 20; or between 0.4 and 0.5, 0.6, 0.7, 0.8, 0.9, 1, 1.5, 2, 2.5, 3, 3.5, 4, 4.5, 5, 6, 7, 8, 9, 10, 11, 12, 13, 14, 15, or 20; or between 0.5 and 0.6, 0.7, 0.8, 0.9, 1, 1.5, 2, 2.5, 3, 3.5, 4, 4.5, 5, 6, 7, 8, 9, 10, 11, 12, 13, 14, 15, or 20; or between 0.6 and 0.7, 0.8, 0.9, 1, 1.5, 2, 2.5, 3, 3.5, 4, 4.5, 5, 6, 7, 8, 9, 10, 11, 12, 13, 14, 15, or 20; or between 0.7 and 0.8, 0.9, 1, 1.5, 2, 2.5, 3, 3.5, 4, 4.5, 5, 6, 7, 8, 9, 10, 11, 12, 13, 14, 15, or 20; or between 0.8 and 0.9, 1, 1.5, 2, 2.5, 3, 3.5, 4, 4.5, 5, 6, 7, 8, 9, 10, 11, 12, 13, 14, 15, or 20; or between 0.9 and 1, 1.5, 2, 2.5, 3, 3.5, 4, 4.5, 5, 6, 7, 8, 9, 10, 11, 12, 13, 14, 15, or 20; or between 1 and 1.5, 2, 2.5, 3, 3.5, 4, 4.5, 5, 6, 7, 8, 9, 10, 11, 12, 13, 14, 15, or 20; or between 1.5 and 2, 2.5, 3, 3.5, 4, 4.5, 5, 6, 7, 8, 9, 10, 11, 12, 13, 14, 15, or 20; or between 2 and 2.5, 3, 3.5, 4, 4.5, 5, 6, 7, 8, 9, 10, 11, 12, 13, 14, 15, or 20; or between 2.5 and 3, 3.5, 4, 4.5, 5, 6, 7, 8, 9, 10, 11, 12, 13, 14, 15, or 20; or between 3 and 3.5, 4, 4.5, 5, 6, 7, 8, 9, 10, 11, 12, 13, 14, 15, or 20; or between 3.5 and 4, 4.5, 5, 6, 7, 8, 9, 10, 11, 12, 13, 14, 15, or 20; or between 4 and 4.5, 5, 6, 7, 8, 9, 10, 11, 12, 13, 14, 15, or 20; or between 4.5 and 5, 6, 7, 8, 9, 10, 11, 12, 13, 14, 15, or 20; or between 5 and 6, 7, 8, 9, 10, 11, 12, 13, 14, 15, or 20; or between 6 and 7, 8, 9, 10, 11, 12, 13, 14, 15, or 20; or between 7 and 8, 9, 10, 11, 12, 13, 14, 15, or 20; or between 8 and 9, 10, 11, 12, 13, 14, 15, or 20; or between 9 and 10, 11, 12, 13, 14, 15, or 20; or between 10 and 11, 12, 13, 14, 15, or 20; or between 11 and 12, 13, 14, 15, or 20; or between 12 and 13, 14, 15, or 20; or between 13 and 14, 15, or 20; or between 14 and 15, or 20; or between 15 and 20; B is between 0.1 and 0.2, 0.3, 0.4, 0.5, 0.6, 0.7, 0.8, 0.9, 1, 1.5, 2, 2.5, 3, 3.5, 4, 4.5, 5, 6, 7, 8, 9, 10, 11, 12, 13, 14, 15, or 20; or between 0.2 and 0.3, 0.4, 0.5, 0.6, 0.7, 0.8, 0.9, 1, 1.5, 2, 2.5, 3, 3.5, 4, 4.5, 5, 6, 7, 8, 9, 10, 11, 12, 13, 14, 15, or 20; or between 0.3 and 0.4, 0.5, 0.6, 0.7, 0.8, 0.9, 1, 1.5, 2, 2.5, 3, 3.5, 4, 4.5, 5, 6, 7, 8, 9, 10, 11, 12, 13, 14, 15, or 20; or between 0.4 and 0.5, 0.6, 0.7, 0.8, 0.9, 1, 1.5, 2, 2.5, 3, 3.5, 4, 4.5, 5, 6, 7, 8, 9, 10, 11, 12, 13, 14, 15, or 20; or between 0.5 and 0.6, 0.7, 0.8, 0.9, 1, 1.5, 2, 2.5, 3, 3.5, 4, 4.5, 5, 6, 7, 8, 9, 10, 11, 12, 13, 14, 15, or 20; or between 0.6 and 0.7, 0.8, 0.9, 1, 1.5, 2, 2.5, 3, 3.5, 4, 4.5, 5, 6, 7, 8, 9, 10, 11, 12, 13, 14, 15, or 20; or between 0.7 and 0.8, 0.9, 1, 1.5, 2, 2.5, 3, 3.5, 4, 4.5, 5, 6, 7, 8, 9, 10, 11, 12, 13, 14, 15, or 20; or between 0.8 and 0.9, 1, 1.5, 2, 2.5, 3, 3.5, 4, 4.5, 5, 6, 7, 8, 9, 10, 11, 12, 13, 14, 15, or 20; or between 0.9 and 1, 1.5, 2, 2.5, 3, 3.5, 4, 4.5, 5, 6, 7, 8, 9, 10, 11, 12, 13, 14, 15, or 20; or between 1 and 1.5, 2, 2.5, 3, 3.5, 4, 4.5, 5, 6, 7, 8, 9, 10, 11, 12, 13, 14, 15, or 20; or between 1.5 and 2, 2.5, 3, 3.5, 4, 4.5, 5, 6, 7, 8, 9, 10, 11, 12, 13, 14, 15, or 20; or between 2 and 2.5, 3, 3.5, 4, 4.5, 5, 6, 7, 8, 9, 10, 11, 12, 13, 14, 15, or 20; or between 2.5 and 3, 3.5, 4, 4.5, 5, 6, 7, 8, 9, 10, 11, 12, 13, 14, 15, or 20; or between 3 and 3.5, 4, 4.5, 5, 6, 7, 8, 9, 10, 11, 12, 13, 14, 15, or 20; or between 3.5 and 4, 4.5, 5, 6, 7, 8, 9, 10, 11, 12, 13, 14, 15, or 20; or between 4 and 4.5, 5, 6, 7, 8, 9, 10, 11, 12, 13, 14, 15, or 20; or between 4.5 and 5, 6, 7, 8, 9, 10, 11, 12, 13, 14, 15, or 20; or between 5 and 6, 7, 8, 9, 10, 11, 12, 13, 14, 15, or 20; or between 6 and 7, 8, 9, 10, 11, 12, 13, 14, 15, or 20; or between 7 and 8, 9, 10, 11, 12, 13, 14, 15, or 20; or between 8 and 9, 10, 11, 12, 13, 14, 15, or 20; or between 9 and 10, 11, 12, 13, 14, 15, or 20; or between 10 and 11, 12, 13, 14, 15, or 20; or between 11 and 12, 13, 14, 15, or 20; or between 12 and 13, 14, 15, or 20; or between 13 and 14, 15, or 20; or between 14 and 15, or 20; or between 15 and 20; C is, where applicable, between 0.1 and 0.2, 0.3, 0.4, 0.5, 0.6, 0.7, 0.8, 0.9, 1, 1.5, 2, 2.5, 3, 3.5, 4, 4.5, 5, 6, 7, 8, 9, 10, 11, 12, 13, 14, 15, or 20; or between 0.2 and 0.3, 0.4, 0.5, 0.6, 0.7, 0.8, 0.9, 1, 1.5, 2, 2.5, 3, 3.5, 4, 4.5, 5, 6, 7, 8, 9, 10, 11, 12, 13, 14, 15, or 20; or between 0.3 and 0.4, 0.5, 0.6, 0.7, 0.8, 0.9, 1, 1.5, 2, 2.5, 3, 3.5, 4, 4.5, 5, 6, 7, 8, 9, 10, 11, 12, 13, 14, 15, or 20; or between 0.4 and 0.5, 0.6, 0.7, 0.8, 0.9, 1, 1.5, 2, 2.5, 3, 3.5, 4, 4.5, 5, 6, 7, 8, 9, 10, 11, 12, 13, 14, 15, or 20; or between 0.5 and 0.6, 0.7, 0.8, 0.9, 1, 1.5, 2, 2.5, 3, 3.5, 4, 4.5, 5, 6, 7, 8, 9, 10, 11, 12, 13, 14, 15, or 20; or between 0.6 and 0.7, 0.8, 0.9, 1, 1.5, 2, 2.5, 3, 3.5, 4, 4.5, 5, 6, 7, 8, 9, 10, 11, 12, 13, 14, 15, or 20; or between 0.7 and 0.8, 0.9, 1, 1.5, 2, 2.5, 3, 3.5, 4, 4.5, 5, 6, 7, 8, 9, 10, 11, 12, 13, 14, 15, or 20; or between 0.8 and 0.9, 1, 1.5, 2, 2.5, 3, 3.5, 4, 4.5, 5, 6, 7, 8, 9, 10, 11, 12, 13, 14, 15, or 20; or between 0.9 and 1, 1.5, 2, 2.5, 3, 3.5, 4, 4.5, 5, 6, 7, 8, 9, 10, 11, 12, 13, 14, 15, or 20; or between 1 and 1.5, 2, 2.5, 3, 3.5, 4, 4.5, 5, 6, 7, 8, 9, 10, 11, 12, 13, 14, 15, or 20; or between 1.5 and 2, 2.5, 3, 3.5, 4, 4.5, 5, 6, 7, 8, 9, 10, 11, 12, 13, 14, 15, or 20; or between 2 and 2.5, 3, 3.5, 4, 4.5, 5, 6, 7, 8, 9, 10, 11, 12, 13, 14, 15, or 20; or between 2.5 and 3, 3.5, 4, 4.5, 5, 6, 7, 8, 9, 10, 11, 12, 13, 14, 15, or 20; or between 3 and 3.5, 4, 4.5, 5, 6, 7, 8, 9, 10, 11, 12, 13, 14, 15, or 20; or between 3.5 and 4, 4.5, 5, 6, 7, 8, 9, 10, 11, 12, 13, 14, 15, or 20; or between 4 and 4.5, 5, 6, 7, 8, 9, 10, 11, 12, 13, 14, 15, or 20; or between 4.5 and 5, 6, 7, 8, 9, 10, 11, 12, 13, 14, 15, or 20; or between 5 and 6, 7, 8, 9, 10, 11, 12, 13, 14, 15, or 20; or between 6 and 7, 8, 9, 10, 11, 12, 13, 14, 15, or 20; or between 7 and 8, 9, 10, 11, 12, 13, 14, 15, or 20; or between 8 and 9, 10, 11, 12, 13, 14, 15, or 20; or between 9 and 10, 11, 12, 13, 14, 15, or 20; or between 10 and 11, 12, 13, 14, 15, or 20; or between 11 and 12, 13, 14, 15, or 20; or between 12 and 13, 14, 15, or 20; or between 13 and 14, 15, or 20; or between 14 and 15, or 20; or between 15 and 20; and D is, where applicable, between 0.1 and 0.2, 0.3, 0.4, 0.5, 0.6, 0.7, 0.8, 0.9, 1, 1.5, 2, 2.5, 3, 3.5, 4, 4.5, 5, 6, 7, 8, 9, 10, 11, 12, 13, 14, 15, or 20; or between 0.2 and 0.3, 0.4, 0.5, 0.6, 0.7, 0.8, 0.9, 1, 1.5, 2, 2.5, 3, 3.5, 4, 4.5, 5, 6, 7, 8, 9, 10, 11, 12, 13, 14, 15, or 20; or between 0.3 and 0.4, 0.5, 0.6, 0.7, 0.8, 0.9, 1, 1.5, 2, 2.5, 3, 3.5, 4, 4.5, 5, 6, 7, 8, 9, 10, 11, 12, 13, 14, 15, or 20; or between 0.4 and 0.5, 0.6, 0.7, 0.8, 0.9, 1, 1.5, 2, 2.5, 3, 3.5, 4, 4.5, 5, 6, 7, 8, 9, 10, 11, 12, 13, 14, 15, or 20; or between 0.5 and 0.6, 0.7, 0.8, 0.9, 1, 1.5, 2, 2.5, 3, 3.5, 4, 4.5, 5, 6, 7, 8, 9, 10, 11, 12, 13, 14, 15, or 20; or between 0.6 and 0.7, 0.8, 0.9, 1, 1.5, 2, 2.5, 3, 3.5, 4, 4.5, 5, 6, 7, 8, 9, 10, 11, 12, 13, 14, 15, or 20; or between 0.7 and 0.8, 0.9, 1, 1.5, 2, 2.5, 3, 3.5, 4, 4.5, 5, 6, 7, 8, 9, 10, 11, 12, 13, 14, 15, or 20; or between 0.8 and 0.9, 1, 1.5, 2, 2.5, 3, 3.5, 4, 4.5, 5, 6, 7, 8, 9, 10, 11, 12, 13, 14, 15, or 20; or between 0.9 and 1, 1.5, 2, 2.5, 3, 3.5, 4, 4.5, 5, 6, 7, 8, 9, 10, 11, 12, 13, 14, 15, or 20; or between 1 and 1.5, 2, 2.5, 3, 3.5, 4, 4.5, 5, 6, 7, 8, 9, 10, 11, 12, 13, 14, 15, or 20; or between 1.5 and 2, 2.5, 3, 3.5, 4, 4.5, 5, 6, 7, 8, 9, 10, 11, 12, 13, 14, 15, or 20; or between 2 and 2.5, 3, 3.5, 4, 4.5, 5, 6, 7, 8, 9, 10, 11, 12, 13, 14, 15, or 20; or between 2.5 and 3, 3.5, 4, 4.5, 5, 6, 7, 8, 9, 10, 11, 12, 13, 14, 15, or 20; or between 3 and 3.5, 4, 4.5, 5, 6, 7, 8, 9, 10, 11, 12, 13, 14, 15, or 20; or between 3.5 and 4, 4.5, 5, 6, 7, 8, 9, 10, 11, 12, 13, 14, 15, or 20; or between 4 and 4.5, 5, 6, 7, 8, 9, 10, 11, 12, 13, 14, 15, or 20; or between 4.5 and 5, 6, 7, 8, 9, 10, 11, 12, 13, 14, 15, or 20; or between 5 and 6, 7, 8, 9, 10, 11, 12, 13, 14, 15, or 20; or between 6 and 7, 8, 9, 10, 11, 12, 13, 14, 15, or 20; or between 7 and 8, 9, 10, 11, 12, 13, 14, 15, or 20; or between 8 and 9, 10, 11, 12, 13, 14, 15, or 20; or between 9 and 10, 11, 12, 13, 14, 15, or 20; or between 10 and 11, 12, 13, 14, 15, or 20; or between 11 and 12, 13, 14, 15, or 20; or between 12 and 13, 14, 15, or 20; or between 13 and 14, 15, or 20; or between 14 and 15, or 20; or between 15 and 20. In some embodiments, A, B, and/or C is within rounding of any of these values (e.g., A is between 0.45 and 0.54, etc.).

Thus, in some aspects of the invention assessing (e.g., detecting, diagnosing) HRD in a sample comprises (1) determining an LOH Region Score for the sample; (2) determining a TAI Region Score for the sample; and (3)(a) detecting (or diagnosing) HRD in the sample based at least in part on a combination of the LOH Region Score and the TAI Region Score (e.g., a Combined CA Region Score) exceeding a reference; or optionally (3)(b) detecting (or diagnosing) an absence of HRD in the sample based at least in part on a combination of the LOH Region Score and the TAI Region Score (e.g., a Combined CA Region Score) not exceeding a reference. In other aspects of the invention assessing (e.g., detecting, diagnosing) HRD in a sample comprises (1) determining an LOH Region Score for the sample; (2) determining an LST Region Score for the sample; and (3)(a) detecting (or diagnosing) HRD in the sample based at least in part on a combination of the LOH Region Score and the LST Region Score (e.g., a Combined CA Region Score) exceeding a reference; or optionally (3)(b) detecting (or diagnosing) an absence of HRD in the sample based at least in part on a combination of the LOH Region Score and the LST Region Score (e.g., a Combined CA Region Score) not exceeding a reference. In other aspects of the invention assessing (e.g., detecting, diagnosing) HRD in a sample comprises (1) determining a TAI Region Score for the sample; (2) determining an LST Region Score for the sample; and (3)(a) detecting (or diagnosing) HRD in the sample based at least in part on a combination of the TAI Region Score and the LST Region Score (e.g., a Combined CA Region Score) exceeding a reference; or optionally (3)(b) detecting (or diagnosing) an absence of HRD in the sample based at least in part on a combination of the TAI Region Score and the LST Region Score (e.g., a Combined CA Region Score) not exceeding a reference. In other aspects of the invention assessing (e.g., detecting, diagnosing) HRD in a sample comprises (1) determining an LOH Region Score for the sample; (2) determining a TAI Region Score for the sample; (3) determining an LST Region Score for the sample; and (4)(a) detecting (or diagnosing) HRD in the sample based at least in part on a combination of the LOH Region Score, the TAI Region Score and the LST Region Score (e.g., a Combined CA Region Score) exceeding a reference; or optionally (4)(b) detecting (or diagnosing) an absence of HRD in the sample based at least in part on the LOH Region Score, the TAI Region Score and the LST Region Score (e.g., a Combined CA Region Score) not exceeding a reference.

In other aspects of the invention assessing (e.g., detecting, diagnosing) HRD in a sample comprises (1) determining the total number of LOH Regions of a certain size or character (e.g., “Indicator LOH Regions”, as defined herein) in the sample; (2) determining the total number of TAI Regions of a certain size or character (e.g., “Indicator TAI Regions”, as defined herein) in the sample; (3) determining the total number of LST Regions of a certain size or character (e.g., “Indicator LST Regions”, as defined herein) in the sample; (4) calculating the average (e.g., arithmetic mean) of the determinations made in (1), (2), and (3); and (5) assessing HRD in the sample based at least in part on the calculated average (e.g., arithmetic mean) made in (4). The above aspects will additionally comprise identifying (or diagnosing) a patient as having an increased likelihood of responding to treatment comprising niraparib (and/or recommending, prescribing or administering a treatment comprising niraparib in the patient).

In some embodiments, the reference (or index) discussed above for the CA Region Score (e.g., the number of Indicator CA Regions) may be 5, 6, 7, 8, 9, 10, 11, 12, 13, 14, 15, 16, 18, 19, 20 or greater, preferably 5, preferably 8, more preferably 9 or 10, most preferably 10. The reference for the total (e.g., combined) length of Indicator CA Regions may be about 75, 90, 105, 120, 130, 135, 150, 175, 200, 225, 250, 275, 300, 325 350, 375, 400, 425, 450, 475, 500 megabases or greater, preferably about 75 megabases or greater, preferably about 90 or 105 megabases or greater, more preferably about 120 or 130 megabases or greater, and more preferably about 135 megabases or greater, and most preferably about 150 megabases or greater. In some embodiments, the reference discussed above for the Combined CA Region Score (e.g., the combined number of Indicator LOH Regions, Indicator, TAI Regions and/or Indicator LST Regions) may be 5, 6, 7, 8, 9, 10, 11, 12, 13, 14, 15, 16, 18, 19, 20, 22, 24, 26, 28, 30, 32, 34, 36, 38, 40, 42, 44, 46, 48, 50 or greater, preferably 5, preferably 10, preferably 15, preferably 20, preferably 25, preferably 30, preferably 35, preferably 40-44, most preferably 42. The reference for the total (e.g., combined) length of Indicator LOH Regions, Indicator TAI Regions and/or Indicator LST Regions may be about 75, 90, 105, 120, 130, 135, 150, 175, 200, 225, 250, 275, 300, 325 350, 375, 400, 425, 450, 475, 500 megabases or greater, preferably about 75 megabases or greater, preferably about 90 or 105 megabases or greater, more preferably about 120 or 130 megabases or greater, and more preferably about 135 megabases or greater, and most preferably about 150 megabases or greater.

In some embodiments, the method comprises detecting an HRD signature in a sample. Thus, in some aspects of the invention detecting an HRD signature in a sample comprises (1) determining the total number of LOH Regions of a certain size or character (e.g., “Indicator LOH Regions”, as defined herein) in the sample; (2) determining the total number of TAI Regions of a certain size or character (e.g., “Indicator TAI Regions”, as defined herein) in the sample; (3) determining the total number of LST Regions of a certain size or character (e.g., “Indicator LST Regions”, as defined herein) in the sample; (4) combining the determinations made in (1), (2), and (3) (e.g., calculating or deriving a Combined CA Region Score); and (5) characterizing a sample in which the Combined CA Region Score is greater than a reference value as having an HRD signature. In some embodiments, the reference value is 42. Thus, in some embodiments a sample is characterized as having an HRD signature when the reference value is 42. In some embodiments, the reference discussed above for the Combined CA Region Score (e.g., the combined number of Indicator LOH Regions, Indicator, TAI Regions and/or Indicator LST Regions) may be 5, 6, 7, 8, 9, 10, 11, 12, 13, 14, 15, 16, 18, 19, 20, 22, 24, 26, 28, 30, 32, 34, 36, 38, 40, 42, 44, 46, 48, 50 or greater, preferably 5, preferably 10, preferably 15, preferably 20, preferably 25, preferably 30, preferably 35, preferably 40-44, most preferably ≧42.

In some embodiments, the number of Indicator CA Regions (or the combined length, a CA Region Score or a Combined CA Region Score) in a sample is considered “greater” than a reference if it is at least 2-, 3-, 4-, 5-, 6-, 7-, 8-, 9-, or 10-fold greater than the reference while in some embodiments, it is considered “greater” if it is at least 1, 2, 3, 4, 5, 6, 7, 8, 9, or 10 standard deviations greater than the reference. Conversely, in some embodiments the number of Indicator CA Regions (or the combined length, a CA Region Score or a Combined CA Region Score) in a sample is considered “not greater” than a reference if it is not more than 2-, 3-, 4-, 5-, 6-, 7-, 8-, 9-, or 10-fold greater than the reference while in some embodiments, it is considered “not greater” if it is not more than 1, 2, 3, 4, 5, 6, 7, 8, 9, or 10 standard deviations greater than the reference.

In some embodiments the reference number (or length, value or score) is derived from a relevant reference population. Such reference populations may include patients (a) with the same cancer as the patient being tested, (b) with the same cancer sub-type, (c) with cancer having similar genetic or other clinical or molecular features, (d) who responded to a particular treatment, (e) who did not respond to a particular treatment, (f) who are apparently healthy (e.g., do not have any cancer or at least do not have the tested patient's cancer), etc. The reference number (or length, value or score) may be (a) representative of the number (or length, value or score) found in the reference population as a whole, (b) an average (mean, median, etc.) of the number (or length, value or score) found in the reference population as a whole or a particular sub-population, (c) representative of the number (or length, value or score) (e.g., an average such as mean or median) found in terciles, quartiles, quintiles, etc. of the reference population as ranked by (i) their respective number (or length, value or score) or (ii) the clinical feature they were found to have (e.g., strength of response, prognosis (including time to cancer-specific death), etc.), or (d) selected to have a high sensitivity for detecting HRD for predicting response to a particular therapy (e.g., platinum, PARP inhibitor, etc.).

In some embodiments the reference or index that, if exceeded by the test value or score from the sample, indicates HRD is the same as the reference that, if not exceeded by the test value or score from the sample, indicates the absence of HRD (or functional HDR). In some embodiments they are different.

As discussed above, this invention provides a method of predicting a cancer patient's response to a cancer treatment regimen comprising niraparib. Thus, in some embodiments an in vitro method of predicting a patient response to a cancer treatment regimen comprising niraparib is provided. The method comprises (1) determining in a patient sample the number of Indicator CA Regions comprising at least two types chosen from Indicator LOH Regions, Indicator TAI Regions, or Indicator LST Regions in at least two pairs of human chromosomes of a breast cancer cell; and (2) diagnosing a patient in whose sample said number of Indicator LOH Regions, Indicator TAI Regions, and/or Indicator LST Regions is greater than a reference number as having an increased likelihood of responding to said cancer treatment regimen. In some embodiments, the reference number is 42.

In some embodiments, the patients are treatment naïve patients. In another aspect, this invention provides a method of treating cancer. Such method is analogous to the methods described above and differs in that a particular treatment regimen is administered (recommended, prescribed, etc.) based at least in part on the determination of CA Regions, LOH Regions, TAI Regions, LST Regions, or scores incorporating these.

In another aspect, this invention features the use of niraparib in the manufacture of a medicament useful for treating a cancer in a patient identified as having (or as having had) a cancer cell determined to have high levels of HRD (e.g., an HRD signature) as described herein.

In another aspect, this document features a method for determining if a patient is likely to respond to a cancer treatment regimen comprising niraparib. The method comprises an in vitro method of predicting patient response to a cancer treatment regimen comprising niraparib. The method comprises (1) determining in a patient sample the number of Indicator CA Regions comprising at least two types chosen from Indicator LOH Regions, Indicator TAI Regions, or Indicator LST Regions in at least two pairs of human chromosomes of a breast cancer cell; (2) providing a test value derived from the number of said Indicator CA Regions; (3) comparing said test value to one or more reference values derived from the number of said Indicator CA Regions in a reference population; and (4) diagnosing a patient in whose sample said test value is greater than said one or more reference numbers as having an increased likelihood of responding to said cancer treatment regimen.

In another aspect, this document features a method for assessing a patient for a likelihood to respond to a cancer treatment regimen comprising niraparib. The method comprises, or consists essentially of, (a) determining that the patient comprises cancer cells having an HRD signature, wherein the presence of more than a reference number of Indicator CA Regions in at least one pair of human chromosomes of a cancer cell of the cancer patient indicates that the cancer cells have the HRD signature, and (b) diagnosing, based at least in part on the presence of the HRD signature, the patient as being likely to respond to the cancer treatment regimen. In another aspect, this document features a method for assessing a patient for a likelihood to respond to a cancer treatment regimen comprising niraparib. The method comprises, or consists essentially of, (a) determining that the patient comprises cancer cells having an HRD signature, wherein the presence of more than a reference number of Indicator CA Regions in at least one pair of human chromosomes of a cancer cell of the cancer patient indicates that the cancer cells have an HRD signature, and (b) diagnosing, based at least in part on the presence of the HRD signature, the patient as being likely to respond to the cancer treatment regimen.

In another aspect, this document features a method for performing a diagnostic analysis of a cancer cell of a patient. The method comprises (1) providing a tumor sample from a patient comprising nucleic acid; (2) determining in a patient sample the number of Indicator CA Regions comprising at least two types chosen from Indicator LOH Regions, Indicator TAI Regions, or Indicator LST Regions in at least two pairs of human chromosomes of a breast cancer cell; (3) providing a test value derived from the number of said Indicator CA Regions; and (4) comparing said test value to one or more reference values derived from the number of said Indicator CA Regions in a reference population; and (5) identifying the patient in whose sample said test value is greater than said one or more reference numbers as having an increased likelihood of responding to said cancer treatment regimen wherein said cancer treatment regimen comprises niraparib. In another aspect, this document features a method for performing a diagnostic analysis of a cancer cell of a patient to determine if the cancer patient is likely to respond to a cancer treatment regimen comprising niraparib. The method comprises, or consists essentially of, (a) detecting the presence of more than a reference number of Indicator CA Regions in at least one pair of human chromosomes of the cancer cell, and (b) identifying or classifying the patient as being likely to respond to the cancer treatment regimen.

In another aspect, this document features a method for diagnosing a patient as being a candidate for a cancer treatment regimen comprising niraparib. The method comprises, or consists essentially of, (a) determining that the patient comprises cancer cells having an HRD signature, wherein the presence of more than a reference number of Indicator CA Regions in at least one pair of human chromosomes of a cancer cell of the cancer patient indicates that the cancer cells have the HRD signature, and (b) diagnosing, based at least in part on the presence of the HRD signature, the patient as being likely to respond to the cancer treatment regimen. In another aspect, this document features a method for diagnosing a patient as being a candidate for a cancer treatment regimen comprising niraparib. The method comprises, or consists essentially of, (a) determining that the patient comprises cancer cells having high an HRD signature, wherein the presence of more than a reference number of Indicator CA Regions in at least one pair of human chromosomes of a cancer cell of the cancer patient indicates that the cancer cells have an HRD signature, and (b) diagnosing, based at least in part on the presence of the HRD signature, the patient as being likely to respond to the cancer treatment regimen.

In another aspect, this invention features the use of a plurality of oligonucleotides capable of hybridizing to a plurality of polymorphic regions of human genomic DNA, in the manufacture of a diagnostic kit useful for determining the total number or combined length of CA Regions in at least a chromosome pair (or DNA derived therefrom) in a sample obtained from a cancer patient, and for detecting an increased likelihood that the cancer patient will respond to a cancer treatment regimen comprising niraparib.

In another aspect, this invention features a system for predicting response to treatment comprising niraparib. The system comprises, or consists essentially of, (a) a sample analyzer configured to produce a plurality of signals about genomic DNA of at least one pair of human chromosomes (or DNA derived therefrom) in the sample, (b) a computer sub-system programmed to calculate, based on the plurality of signals, the number or combined length of CA Regions in the at least one pair of human chromosomes, and (c) a computer sub-system programmed to assess (and optionally display) the likelihood the patient from whom the sample is derived will respond to treatment comprising niraparib. The computer sub-system can be programmed to compare the number or combined length of CA Regions to a reference number to detect an increased likelihood that the cancer patient will respond to a cancer treatment regimen comprising niraparib. The system can comprise an output module configured to display (a), (b), or (c). The system can comprise an output module configured to display a recommendation for the use of the cancer treatment regimen.

In another aspect, the invention provides a computer program product embodied in a computer readable medium that, when executing on a computer, provides instructions for (a) detecting the presence or absence of any CA Region along one or more of human chromosomes (e.g., other than the human X and Y sex chromosomes) (the CA Regions optionally being Indicator CA Regions); (b) determining the total number or combined length of the CA Regions in the one or more chromosome pairs and (c) assessing (and optionally display) the likelihood the patient from whom the sample is derived will respond to treatment comprising niraparib. The computer program product can include other instructions.

In another aspect, the present invention provides a diagnostic kit. The kit comprises, or consists essentially of, at least 500 oligonucleotides capable of hybridizing to a plurality of polymorphic regions of human genomic DNA (or DNA derived therefrom); and a computer program product as described in the preceding paragraph. The computer program product can include other instructions.

In some embodiments of any one or more of the aspects of the invention described in the preceding paragraphs, any one or more of the following can be applied as appropriate. The CA Regions can be determined in at least two, five, ten, or 21 pairs of human chromosomes. The cancer cell can be an ovarian, breast, lung or esophageal cancer cell. The reference can be 6, 7, 8, 9, 10, 11, 12, 13, 14, 15, 16, 17, 18 or 20 or greater. The at least one pair of human chromosomes can exclude human chromosome 17. The patient can be a treatment naïve patient.

As described herein, a sample (e.g., cancer cell sample or a sample containing DNA derived from one or more cancer cells) can be identified as having an “HRD signature” (or alternatively called “HDR-deficiency signature”) if the genome of the cells being assessed contains (a) any of an LOH Region Score, a TAI Region Score or an LST Region Score exceeding a reference or (b) a Combined CA Region Score exceeding a reference. Conversely, a sample (e.g., cancer cell sample or a sample containing DNA derived from one or more cancer cells) can be identified as lacking an “HRD signature” (or alternatively called “HDR-deficiency signature”) if the genome of the cells being assessed contains (a) an LOH Region Score, a TAI Region Score and an LST Region Score each not exceeding a reference or (b) a Combined CA Region Score not exceeding a reference.

Cells (e.g., cancer cells) identified as having an HRD signature can be classified as having an increased likelihood of having an HDR deficiency and/or as having an increased likelihood of having a deficient status in one or more genes in the HDR pathway, which can in turn be used to assess the patient's likelihood of response to niraparib as described herein. For example, cancer cells identified as having an HRD signature can be classified as having an increased likelihood of having an HDR deficient status.

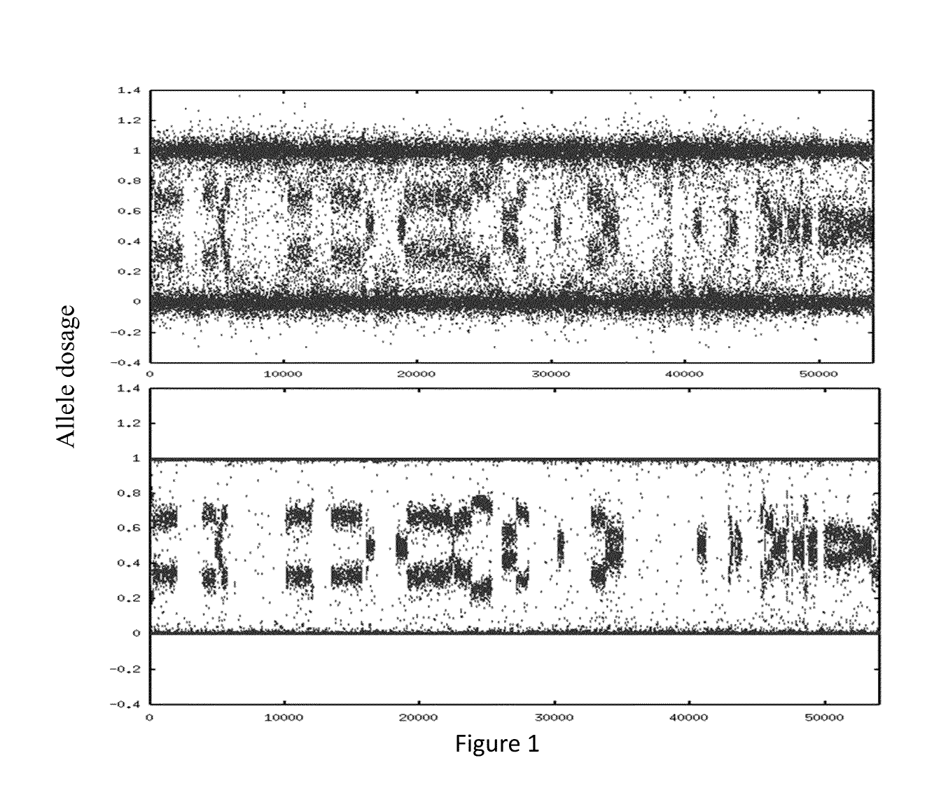

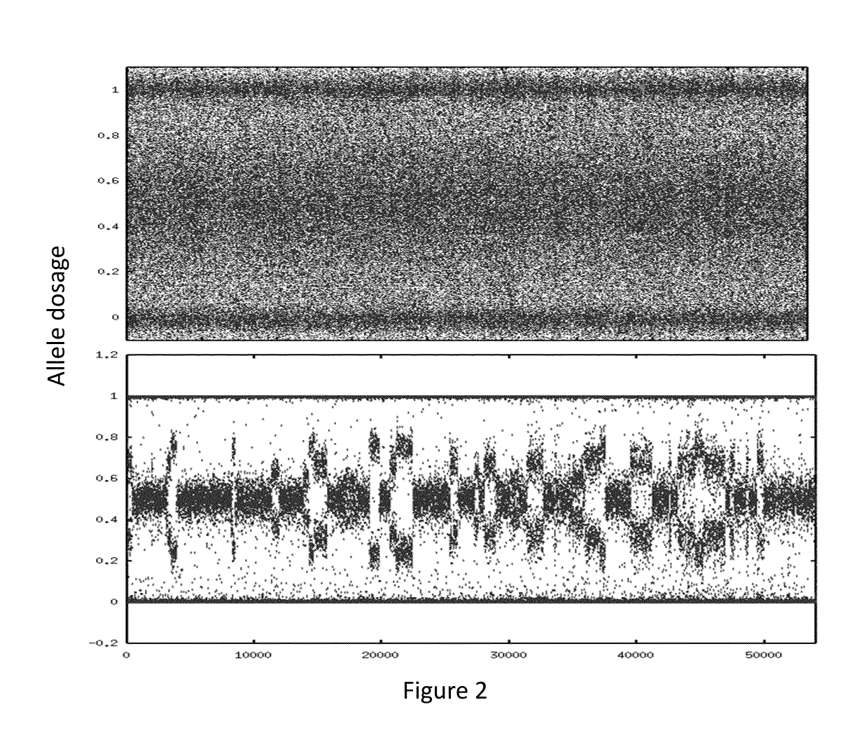

As described herein, identifying CA loci (as well as the size and number of CA Regions) can include, first, determining the genotype of a sample at various genomic loci (e.g., SNP loci, individual bases in large-scale sequencing) and, second, determining whether the loci exhibit any of LOH, TAI or LST. Any appropriate technique can be used to determine genotypes at loci of interest within the genome of a cell. For example, single nucleotide polymorphism (SNP) arrays (e.g., human genome-wide SNP arrays), targeted sequencing of loci of interest (e.g., sequencing SNP loci and their surrounding sequences), and even large-scale sequencing (e.g., whole exome, transcriptome, or genome sequencing) can be used to identify loci as being homozygous or heterozygous. A nucleic acid signature for targeted loci of interest used to determine HRD can include additional mechanisms known in the art, e.g., gene mutations, large rearrangements, methylation status, and combinations thereof. Typically, an analysis of the homozygous or heterozygous nature of loci over a length of a chromosome can be performed to determine the length of CA Regions. For example, a stretch of SNP locations that are spaced apart (e.g., spaced about 25 kb to about 100 kb apart) along a chromosome can be evaluated using SNP array results to determine not only the presence of a region of homozygosity (e.g., LOH) along a chromosome but also the length of that region. Results from a SNP array can be used to generate a graph that plots allele dosages along a chromosome. Allele dosage dᵢ for SNP i can be calculated from adjusted signal intensities of two alleles (Aᵢ and Bᵢ): dᵢ=Aᵢ/(Aᵢ+Bᵢ). An example of such a graph is presented in FIGS. 1 and 2, which show the difference between fresh frozen and FFPE samples and between SNP microarray and SNP sequencing analyses. Numerous variations on nucleic acid arrays useful in the invention are known in the art. These include the arrays used in the various examples below (e.g., Affymetrix 500K GeneChip array in Example 3; Affymetrix OncoScan™ FFPE Express 2.0 Services (Formerly MIP CN Services) in Example 4).

Once a sample's genotype has been determined for a plurality of loci (e.g., SNPs), common techniques can be used to identify loci and regions of LOH, TAI and LST, including those described in International Application nos. PCT/US2011/040953 (published as WO/2011/160063), PCT/US2011/048427 (published as WO/2012/027224), PCT/EP2013/061707 (published as WO/2013/182645), and PCT/US2014/033014 (published as WO/2014/165785), the contents of each of which are hereby incorporated by reference in their entirety. In some embodiments determining whether chromosomal imbalance or large scale transitions includes determining whether these are somatic or germline aberrations. One way to determine to do this is to compare the somatic genotype to the germline. For example, the genotype for a plurality of loci (e.g., SNPs) can be determined in both a germline (e.g., blood) sample and a somatic (e.g., tumor) sample. The genotypes for each sample can be compared (typically computationally) to determine where the genome of the germline cell was heterozygous and the genome of the somatic cell is homozygous. Such loci are LOH loci and regions of such loci are LOH Regions.

Computational techniques can also be used to determine whether an aberration is germline or somatic. Such techniques are particularly useful when a germline sample is not available for analysis and comparison. For example, algorithms such as those described elsewhere can be used to detect LOH regions using information from SNP arrays (Nannya et al., Cancer Res. (2005) 65:6071-6079 (2005)). Typically these algorithms do not explicitly take into account contamination of tumor samples with benign tissue. Cf. International Application No. PCT/US2011/026098 to Abkevich et al.; Goransson et al., PLoS One (2009) 4(6):e6057. This contamination is often high enough to make the detection of LOH regions challenging. Improved analytical methods according to the present invention for identifying LOH, TAI and LST, even in spite of contamination, include those embodied in computer software products as described below.

The following is one example. If the observed ratio of the signals of two alleles, A and B, is two to one, there are two possibilities. The first possibility is that cancer cells have LOH with deletion of allele B in a sample with 50% contamination with normal cells. The second possibility is that there is no LOH but allele A is duplicated in a sample with no contamination with normal cells. An algorithm can be implemented as a computer program as described herein to reconstruct LOH regions based on genotype (e.g., SNP genotype) data. One point of the algorithm is to first reconstruct allele specific copy numbers (ASCN) at each locus (e.g., SNP). ASCNs are the numbers of copies of both paternal and maternal alleles. An LOH region is then determined as a stretch of SNPs with one of the ASCNs (paternal or maternal) being zero. The algorithm can be based on maximizing a likelihood function and can be conceptually akin to a previously described algorithm designed to reconstruct total copy number (rather than ASCN) at each locus (e.g., SNP). See International Application No. PCT/US2011/026098 to Abkevich et al. The likelihood function can be maximized over ASCN of all loci, level of contamination with benign tissue, total copy number averaged over the whole genome, and sample specific noise level. The input data for the algorithm can include or consist of (1) sample-specific normalized signal intensities for both allele of each locus and (2) assay-specific (specific for different SNP arrays and for sequence based approach) set of parameters defined based on analysis of large number of samples with known ASCN profiles.

In some cases, nucleic acid sequencing techniques can be used to genotype loci. For example, genomic DNA from a cell sample (e.g., a cancer cell sample) can be extracted and fragmented. Any appropriate method can be used to extract and fragment genomic nucleic acid including, without limitation, commercial kits such as QIAamp™ DNA Mini Kit (Qiagen™), MagNA™ Pure DNA Isolation Kit (Roche Applied Science™) and GenElute™ Mammalian Genomic DNA Miniprep Kit (Sigma-Aldrich™). Once extracted and fragmented, either targeted or untargeted sequencing can be done to determine the sample's genotypes at loci. For example, whole genome, whole transcriptome, or whole exome sequencing can be done to determine genotypes at millions or even billions of base pairs (i.e., base pairs can be “loci” to be evaluated).

In some cases, targeted sequencing of known polymorphic loci (e.g., SNPs and surrounding sequences) can be done as an alternative to microarray analysis. For example, the genomic DNA can be enriched for those fragments containing a locus (e.g., SNP location) to be analyzed using kits designed for this purpose (e.g., Agilent SureSelect™, Illumina TruSeq Capture™, and Nimblegen SeqCap EZ Choice™). For example, genomic DNA containing the loci to be analyzed can be hybridized to biotinylated capture RNA fragments to form biotinylated RNA/genomic DNA complexes. Alternatively, DNA capture probes may be utilized resulting in the formation of biotinylated DNA/genomic DNA hybrids. Streptavidin coated magnetic beads and a magnetic force can be used to separate the biotinylated RNA/genomic DNA complexes from those genomic DNA fragments not present within a biotinylated RNA/genomic DNA complex. The obtained biotinylated RNA/genomic DNA complexes can be treated to remove the captured RNA from the magnetic beads, thereby leaving intact genomic DNA fragments containing a locus to be analyzed. These intact genomic DNA fragments containing the loci to be analyzed can be amplified using, for example, PCR techniques. The amplified genomic DNA fragments can be sequenced using a high-throughput sequencing technology or a next-generation sequencing technology such as Illumina HiSeq™, Illumina MiSeq™, Life Technologies SoLID™ or Ion Torrent™, or Roche 454™.

The sequencing results from the genomic DNA fragments can be used to identify loci as exhibiting or not exhibiting a CA, analogous to the microarray analysis described herein. In some cases, an analysis of the genotype of loci over a length of a chromosome can be performed to determine the length of CA Regions. For example, a stretch of SNP locations that are spaced apart (e.g., spaced about 25 kb to about 100 kb apart) along a chromosome can be evaluated by sequencing, and the sequencing results used to determine not only the presence of a CA Region but also the length of that CA Region. Obtained sequencing results can be used to generate a graph that plots allele dosages along a chromosome. Allele dosage dᵢ for SNP i can be calculated from adjusted number of captured probes for two alleles (Aᵢ and Bᵢ): dᵢ=Aᵢ/(Aᵢ+Bᵢ). An example of such a graph is presented in FIGS. 1 and 2. Determining whether an aberration is germline or somatic can be performed as described herein.

In some cases, a certain number of polymorphic loci (e.g., SNPs) can be used in the analysis. In some cases these loci can be randomly spaced in the genome, at least a certain space apart, etc. In some cases, about 40, 50, 60, 70, 80, 90, 100, 110, 120, 130 thousand or more loci across a human genome (or across the chromosomes analyzed) can be used in the analysis. For example, between about 70,000 and about 90,000 (e.g., about 80,000) SNPs can be selected for analysis with a SNP array-based assay, and between about 45,000 and about 55,000 (e.g., about 54,000) SNPs can be selected for analysis with a sequencing-based assay.

As described herein, any appropriate type of sample can be assessed. For example, a sample containing triple negative breast cancer cells can be assessed to determine if the genome of the cancer cells contains an HRD signature that can be used to assess likelihood of response to treatment comprising niraparib. Examples of samples containing cancer cells that can be assessed as described herein include, without limitation, tumor biopsy samples (e.g., breast tumor biopsy samples), formalin-fixed, paraffin-embedded tissue samples containing cancer cells, core needle biopsies, fine needle aspirates, and samples containing cancer cells shed from a tumor (e.g., blood, urine or other bodily fluids). For formalin-fixed, paraffin-embedded tissue samples, the sample can be prepared by DNA extraction using a genomic DNA extraction kit optimized for FFPE tissue, including but not limited to those described above (e.g., QuickExtract™ FFPE DNA Extraction Kit (Epicentre™), and QIAamp™ DNA FFPE Tissue Kit (Qiagen™)).

In some cases, laser dissection techniques can be performed on a tissue sample to minimize the number of non-cancer cells within a cancer cell sample to be assessed. In some cases, antibody based purification methods can be used to enrich for cancer cells and/or deplete non-cancer cells. Examples of antibodies that could be used for cancer cell enrichment include, without limitation, anti-EpCAM, anti-TROP-2, anti-c-Met, anti-Folate binding protein, anti-N-Cadherin, anti-CD318, anti-antimesencymal stem cell antigen, anti-Her2, anti-MUC1, anti-EGFR, anti-cytokeratins (e.g., cytokeratin 7, cytokeratin 20, etc.), anti-Caveolin-1, anti-PSA, anti-CA125, and anti-surfactant protein antibodies.

When assessing the genome of cancer cells for the presence or absence of an HRD signature, one or more (e.g., one, two, three, four, five, six, seven, eight, nine, ten, eleven, twelve, 13, 14, 15, 16, 17, 18, 19, 20, 21, 22, or 23) pairs of chromosomes can be assessed. In some cases, the genome of cancer cells is assessed for the presence or absence of an HRD signature using one or more (e.g., one, two, three, four, five, six, seven, eight, nine, ten, eleven, twelve, 13, 14, 15, 16, 17, 18, 19, 20, 21, 22, 23) pairs of chromosomes.

In some cases, it can be helpful to exclude certain chromosomes from this analysis. For example, in the case of females, a pair to be assessed can include the pair of X sex chromosomes; whereas, in the case of males, a pair of any autosomal chromosomes (i.e., any pair other than the pair of X and Y sex chromosomes) can be assessed. As another example, in some cases the chromosome number 17 pair may be excluded from the analysis. It has been determined that certain chromosomes carry unusually high levels of CA in certain cancers and, thus, it can be helpful to exclude such chromosomes when analyzing samples as described herein from patients having these cancers. In some cases, the sample is from a patient having breast cancer (e.g., triple-negative breast cancer) or ovarian cancer, and the chromosome to be excluded is chromosome 17.

Thus, a predefined number of chromosomes may be analyzed to determine the number of Indicator CA Regions (or the CA Region Score or Combined CA Region Score), preferably the number of CA Regions of a length of greater than 9 megabases, 10 megabases, 12 megabases, 14 megabases, more preferably greater than 15 megabases. Alternatively or in addition, the sizes of all identified Indicator CA Regions may be summed up to obtain a total length of Indicator CA Regions.

As described herein, triple negative breast cancer patients having cancer cells (or samples derived therefrom) identified as having an HRD signature status can be classified, based at least in part on such HRD signature, as being likely to respond to a cancer treatment regimen comprising niraparib. Once classified as being likely to respond to such a cancer treatment regimen, the cancer patient can be treated with such a cancer treatment regimen. In some embodiments, the patients are treatment naïve patients. The invention thus provides a method of treating a patient comprising detecting an HRD signature as described herein and administering (or recommending or prescribing) a treatment regimen comprising niraparib. Any appropriate method for treating the cancer at issue with a regimen comprising niraparib can be used to treat a cancer patient identified as having cancer cells having an HRD signature.

Once treated for a particular period of time (e.g., between one to six months), the patient can be assessed to determine whether or not the treatment regimen has an effect. If a beneficial effect is detected, the patient can continue with the same or a similar cancer treatment regimen. If a minimal or no beneficial effect is detected, then adjustments to the cancer treatment regimen can be made. For example, the dose, frequency of administration, or duration of treatment can be increased. In some cases, additional anti-cancer agents can be added to the treatment regimen or a particular anti-cancer agent can be replaced with one or more different anti-cancer agents. The patient being treated can continue to be monitored as appropriate, and changes can be made to the cancer treatment regimen as appropriate.

As described herein, this document provides methods for assessing patients for cells (e.g., cancer cells) having an HRD signature. In some embodiments, one or more clinicians or medical professionals can determine whether a sample from the patient comprises cancer cells (or whether a sample comprises DNA derived from such cells) having an HRD signature. In some cases, one or more clinicians or medical professionals can determine if a patient contains cancer cells having an HRD signature by obtaining a cancer cell sample from the patient and assessing the DNA of cancer cells of the cancer cell sample to determine the presence or absence of an HRD signature as described herein.

In some cases, one or more clinicians or medical professionals can obtain a cancer cell sample from a patient and provide that sample to a testing laboratory having the ability to assess DNA of cancer cells of the cancer cell sample to provide an indication about the presence or absence of an HRD signature for assessing likelihood of response to niraparib as described herein. In some cases, a clinician or medical professional or group of clinicians or medical professionals can diagnose a patient determined to have cancer cells having an HRD signature as having cancer cells likely to respond to a cancer treatment regimen comprising niraparib. In some embodiments, the patients are treatment naïve patients. Such a diagnosis can be based solely on a determination that a sample from the patient comprises cancer cells (or whether a sample comprises DNA derived from such cells) having an HRD signature or can be based at least in part on a determination that a sample from the patient comprises cancer cells (or whether a sample comprises DNA derived from such cells) having an HRD signature. For example, a patient determined to have cancer cells having an HRD signature can be diagnosed as being likely to respond to such a cancer treatment regimen based on the combination of the presence of an HRD signature and deficient status in BRCA1 and/or BRCA2. As described herein, a patient determined to have cancer cells having an HRD signature can be diagnosed as likely to respond to a cancer treatment regimen that includes the use of niraparib. In some embodiments, the patients are treatment naïve patients.

The results of any analyses according to the invention will often be communicated to physicians, genetic counselors and/or patients (or other interested parties such as researchers) in a transmittable form that can be communicated or transmitted to any of the above parties. Such a form can vary and can be tangible or intangible. The results can be embodied in descriptive statements, diagrams, photographs, charts, images or any other visual forms. For example, graphs or diagrams showing genotype or LOH, TAI or LST (or HRD combined status) information can be used in explaining the results. The statements and visual forms can be recorded on a tangible medium such as papers, computer readable media such as floppy disks, compact disks, flash memory, etc., or in an intangible medium, e.g., an electronic medium in the form of email or website on internet or intranet. In addition, results can also be recorded in a sound form and transmitted through any suitable medium, e.g., analog or digital cable lines, fiber optic cables, etc., via telephone, facsimile, wireless mobile phone, internet phone and the like.

Thus, the information and data on a test result can be produced anywhere in the world and transmitted to a different location. As an illustrative example, when an assay is conducted outside the United States, the information and data on a test result may be generated, cast in a transmittable form as described above, and then imported into the United States. Accordingly, the present invention also encompasses a method for producing a transmittable form of information on an HRD signature for at least one patient sample. The method comprises the steps of (1) determining an HRD signature according to methods of the present invention; and (2) embodying the result of the determining step in a transmittable form. The transmittable form is a product of such a method.

Some embodiments of the invention described herein may involve a step of correlating the presence of an HRD signature according to the present invention (e.g., the total number of Indicator CA Regions or a CA Region Score or Combined CA Region Score greater than a reference) to an increased likelihood of response to a treatment regimen comprising niraparib. Throughout this document, wherever such an embodiment is described, another embodiment of the invention may involve, in addition to or instead of a correlating step, one or both of the following steps: (a) concluding that the patient has the clinical feature based at least in part on the presence or absence of the HRD signature; or (b) communicating that the patient has the clinical feature based at least in part on the presence or absence of the HRD signature.

By way of illustration, but not limitation, one embodiment in this document is a method of predicting a cancer patient's response to a cancer treatment regimen comprising niraparib, said method comprising: (1) determining in a sample two or more of (a) an LOH Region Score for the sample; (b) a TAI Region Score for the sample; or (c) an LST Region Score for the sample; and (2)(a) correlating a combination of two or more of the LOH Region Score, the TAI Region Score and the LST Region Score (e.g., a Combined CA Region Score) exceeding a reference to an increased likelihood of responding to the treatment regimen; or optionally (2)(b) correlating a combination of two or more of the LOH Region Score, the TAI Region Score and the LST Region Score (e.g., a Combined CA Region Score) not exceeding a reference to a not increased likelihood of responding to the treatment regimen; or optionally (2)(c) correlating an average (e.g., arithmetic mean) of the LOH Region Score, the TAI Region Score, and the LST Region Score. According to the preceding paragraph, this description of this embodiment is understood to include a description of two alternative related embodiments. One such embodiment provides a method of predicting a cancer patient's response to a cancer treatment regimen comprising niraparib, said method comprising: (1) determining in a sample two or more of (a) an LOH Region Score for the sample; (b) a TAI Region Score for the sample; or (c) an LST Region Score for the sample; or (d) an average (e.g., arithmetic mean) of the LOH Region Score, the TAI Region Score, and the LST Region Score; and (2)(a) concluding that said patient has an increased likelihood of responding to said cancer treatment regimen based at least in part on a combination of two or more of the LOH Region Score, the TAI Region Score and the LST Region Score (e.g., a Combined CA Region Score) exceeding a reference; or optionally (2)(b) concluding that said patient has a not increased likelihood of responding to said cancer treatment regimen based at least in part on a combination of two or more of the LOH Region Score, the TAI Region Score and the LST Region Score (e.g., a Combined CA Region Score), or an average (e.g., arithmetic mean) of the LOH Region Score, the TAI Region Score, and the LST Region Score, not exceeding a reference. Another such embodiment provides a method of predicting a cancer patient's response to a cancer treatment regimen comprising niraparib, said method comprising: (1) determining in a sample two or more of (a) an LOH Region Score for the sample; (b) a TAI Region Score for the sample; or (c) an LST Region Score for the sample; or (d) an average (e.g., arithmetic mean) of the LOH Region Score, the TAI Region Score, and the LST Region Score; and (2)(a) communicating that said patient has an increased likelihood of responding to said cancer treatment regimen based at least in part on a combination of two or more of the LOH Region Score, the TAI Region Score and the LST Region Score (e.g., a Combined CA Region Score); or an average (e.g., arithmetic mean) of the LOH Region Score, the TAI Region Score, and the LST Region Score, exceeding a reference; or optionally (2)(b) communicating that said patient has a not increased likelihood of responding to said cancer treatment regimen based at least in part on a combination of two or more of the LOH Region Score, the TAI Region Score and the LST Region Score (e.g., a Combined CA Region Score); or an average (e.g., arithmetic mean) of the LOH Region Score, the TAI Region Score, and the LST Region Score, not exceeding a reference.

In each embodiment described in this document involving correlating a particular assay or analysis output (e.g., total number of Indicator CA Regions greater than a reference number, presence of an HRD signature etc.) to some likelihood (e.g., increased, not increased, decreased, etc.) of response to treatment comprising niraparib, or additionally or alternatively concluding or communicating such clinical feature based at least in part on such particular assay or analysis output, such correlating, concluding or communicating may comprise assigning a risk or likelihood of the clinical feature occurring based at least in part on the particular assay or analysis output. In some embodiments, such risk is a percentage probability of the event or outcome occurring. In some embodiments, the patient is assigned to a risk group (e.g., low risk, intermediate risk, high risk, etc.). In some embodiments “low risk” is any percentage probability below 5%, 10%, 15%, 20%, 25%, 30%, 35%, 40%, 45%, or 50%. In some embodiments “intermediate risk” is any percentage probability above 5%, 10%, 15%, 20%, 25%, 30%, 35%, 40%, 45%, or 50% and below 15%, 20%, 25%, 30%, 35%, 40%, 45%, 50%, 55%, 60%, 65%, 70%, or 75%. In some embodiments “high risk” is any percentage probability above 25%, 30%, 35%, 40%, 45%, 50%, 55%, 60%, 65%, 70%, 75%, 80%, 85%, 90%, 95%, or 99%.

As used herein, “communicating” a particular piece of information means to make such information known to another person or transfer such information to a thing (e.g., a computer). In some methods of the invention, a patient's prognosis or likelihood of response to a particular treatment is communicated. In some embodiments, the information used to arrive at such a prognosis or response prediction (e.g., HRD signature according to the present invention, etc.) is communicated. This communication may be auditory (e.g., verbal), visual (e.g., written), electronic (e.g., data transferred from one computer system to another), etc. In some embodiments, communicating a cancer classification (e.g., prognosis, likelihood of response, appropriate treatment, etc.) comprises generating a report that communicates the cancer classification. In some embodiments the report is a paper report, an auditory report, or an electronic record. In some embodiments the report is displayed and/or stored on a computing device (e.g., handheld device, desktop computer, smart device, website, etc.). In some embodiments the cancer classification is communicated to a physician (e.g., a report communicating the classification is provided to the physician). In some embodiments the cancer classification is communicated to a patient (e.g., a report communicating the classification is provided to the patient). Communicating a cancer classification can also be accomplished by transferring information (e.g., data) embodying the classification to a server computer and allowing an intermediary or end-user to access such information (e.g., by viewing the information as displayed from the server, by downloading the information in the form of one or more files transferred from the server to the intermediary or end-user's device, etc.).

Wherever an embodiment of the invention comprises concluding some fact (e.g., a patient's prognosis or a patient's likelihood of response to a particular treatment regimen), this may include in some embodiments a computer program concluding such fact, typically after performing an algorithm that applies information on CA Regions according to the present invention.

In each embodiment described herein involving a number of CA Regions (e.g., Indicator CA Regions), or a total combined length of such CA Regions, or an average (e.g., arithmetic mean) of the combined CAR Region scores, the present invention encompasses a related embodiment involving a test value or score (e.g., CA Region Score, LOH Region Score, etc.) derived from, incorporating, and/or, at least to some degree, reflecting such number or length. In other words, the bare CA Region numbers or lengths need not be used in the various methods, systems, etc. of the invention; a test value or score derived from such numbers or lengths may be used. For example, one embodiment of the invention provides a method of treating cancer in a patient, comprising: (1) determining in a sample from said patient two or more of, or an average (e.g., arithmetic mean) of, (a) the number of Indicator LOH Regions, (b) the number of Indicator TAI Regions, or (c) the number of Indicator LST Regions; (2) providing one or more test values derived from said number of Indicator LOH Regions, Indicator TAI Regions, and/or Indicator LST Regions; (3) comparing said test value(s) to one or more reference values (e.g., reference values derived from the number of Indicator LOH regions, Indicator TAI Regions, and/or Indicator LST Regions in a reference population (e.g., mean, median, terciles, quartiles, quintiles, etc.)); and (4)(a) administering to said patient a treatment comprising niraparib, or recommending or prescribing or initiating such a treatment based at least in part on said comparing step revealing that one or more of the test values is greater (e.g., at least 2-, 3-, 4-, 5-, 6-, 7-, 8-, 9-, or 10-fold greater; at least 1, 2, 3, 4, 5, 6, 7, 8, 9, or 10 standard deviations greater) than at least one said reference value; or optionally (4)(b) recommending or prescribing or initiating a treatment regimen not comprising niraparib based at least in part on said comparing step revealing that one or more of the test values is not greater (e.g., not more than 2-, 3-, 4-, 5-, 6-, 7-, 8-, 9-, or 10-fold greater; not more than 1, 2, 3, 4, 5, 6, 7, 8, 9, or 10 standard deviations greater) than at least one said reference value.

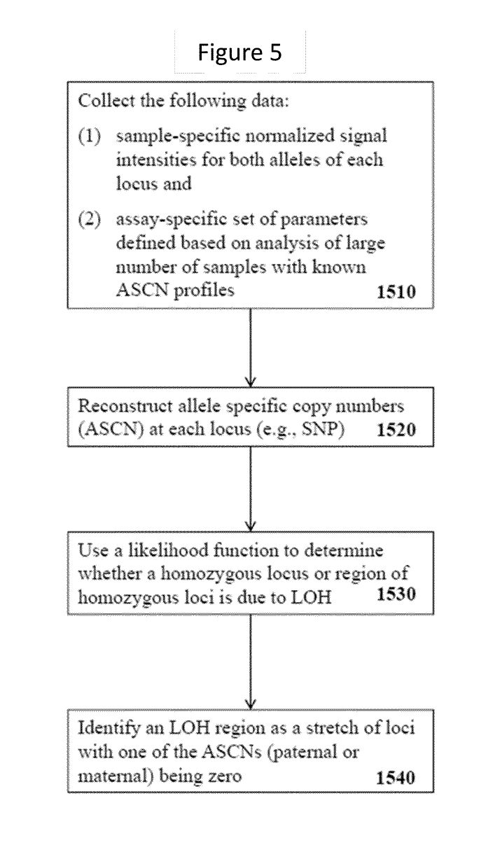

FIG. 5 shows an exemplary process by which a computing system (or a computer program (e.g., software) containing computer-executable instructions) can identify LOH loci or regions from genotype data as described herein. This process may be adapted to use in determining TAI and LST as will be apparent to those skilled in the art. If the observed ratio of the signals of two alleles, A and B, is two to one, there are two possibilities. The first possibility is that cancer cells have LOH with deletion of allele B in a sample with 50% contamination with normal cells. The second possibility is that there is no LOH but allele A is duplicated in a sample with no contamination with normal cells. The process begins at box 1500, where the following data are collected by the computing system; (1) sample-specific normalized signal intensities for both alleles of each locus and (2) assay-specific (specific for different SNP arrays and for sequence based approach) set of parameters defined based on analysis of large number of samples with known ASCN profiles. As described herein, any appropriate assay such as a SNP array-based assay or sequencing-based assay can be used to assess loci along a chromosome for homozygosity or heterozygosity. In some cases, a system including a signal detector and a computer can be used to collect data (e.g., fluorescent signals or sequencing results) regarding the homozygous or heterozygous nature of the plurality of loci (e.g., sample-specific normalized signal intensities for both alleles of each locus). At box 1510, allele specific copy numbers (ASCN) are reconstructed at each locus (e.g., each SNP). ASCNs are the numbers of copies of both paternal and maternal alleles. At box 1530, a likelihood function is used to determine whether a homozygous locus or region of homozygous loci is due to LOH. This can be conceptually analogous to a previously described algorithm designed to reconstruct total copy number (rather than ASCN) at each locus (e.g., SNP). See International Application No. PCT/US2011/026098 to Abkevich et al. The likelihood function can be maximized over ASCN of all loci, level of contamination with benign tissue, total copy number averaged over the whole genome, and sample specific noise level. At box 1540, an LOH region is determined as a stretch of SNPs with one of the ASCNs (paternal or maternal) being zero. In some embodiments, the computer process further comprises a step of inquiring or determining whether a patient is treatment naïve.

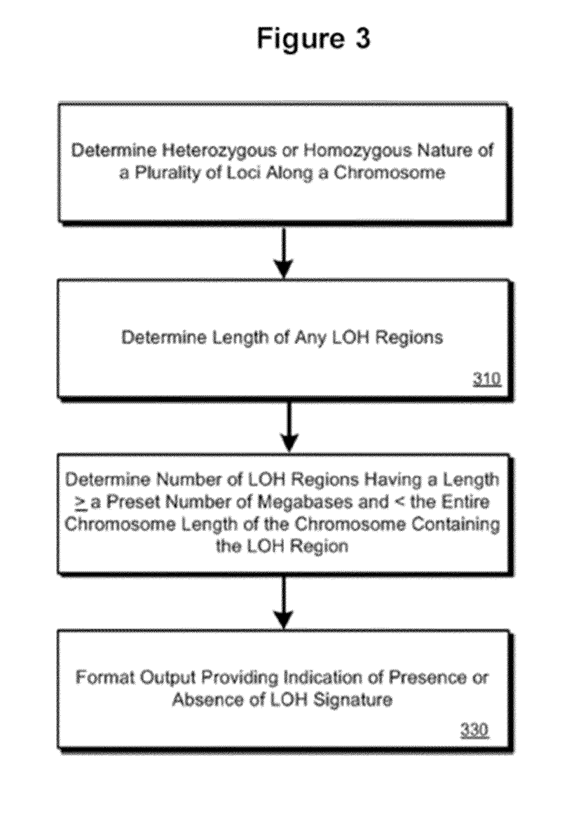

FIG. 3 shows an exemplary process by which a computing system can determine the presence or absence of an LOH signature and is included to illustrate how this process can, as will be apparent to those skilled in the art, be applied to TAI and LST. The process begins at box 300, where data regarding the homozygous or heterozygous nature of a plurality of loci along a chromosome is collected by the computing system. As described herein, any appropriate assay such as a SNP array-based assay or sequencing-based assay can be used to assess loci along a chromosome for homozygosity or heterozygosity. In some cases, a system including a signal detector and a computer can be used to collect data (e.g., fluorescent signals or sequencing results) regarding the homozygous or heterozygous nature of the plurality of loci. At box 310, data regarding the homozygous or heterozygous nature of a plurality of loci as well as the location or spatial relationship of each locus is assessed by the computing system to determine the length of any LOH regions present along a chromosome. At box 320, data regarding the number of LOH regions detected and the length of each detected LOH region is assessed by the computing system to determine the number of LOH regions that have a length (a) greater than or equal to a preset number of Mb (e.g., 15 Mb) and (b) less than the entire length of the chromosome containing that LOH region. Alternatively the computing system can determine the total or combined LOH length as described above. At box 330, the computing system formats an output providing an indication of the presence or absence of an HRD signature. Once formatted, the computing system can present the output to a user (e.g., a laboratory technician, clinician, or medical professional). As described herein, the presence or absence of an HRD signature can be used to provide an indication about a patient's likely HDR status, an indication about the likely presence or absence of genetic mutations in genes of the HDR pathway, and/or an indication about possible cancer treatment regimens.

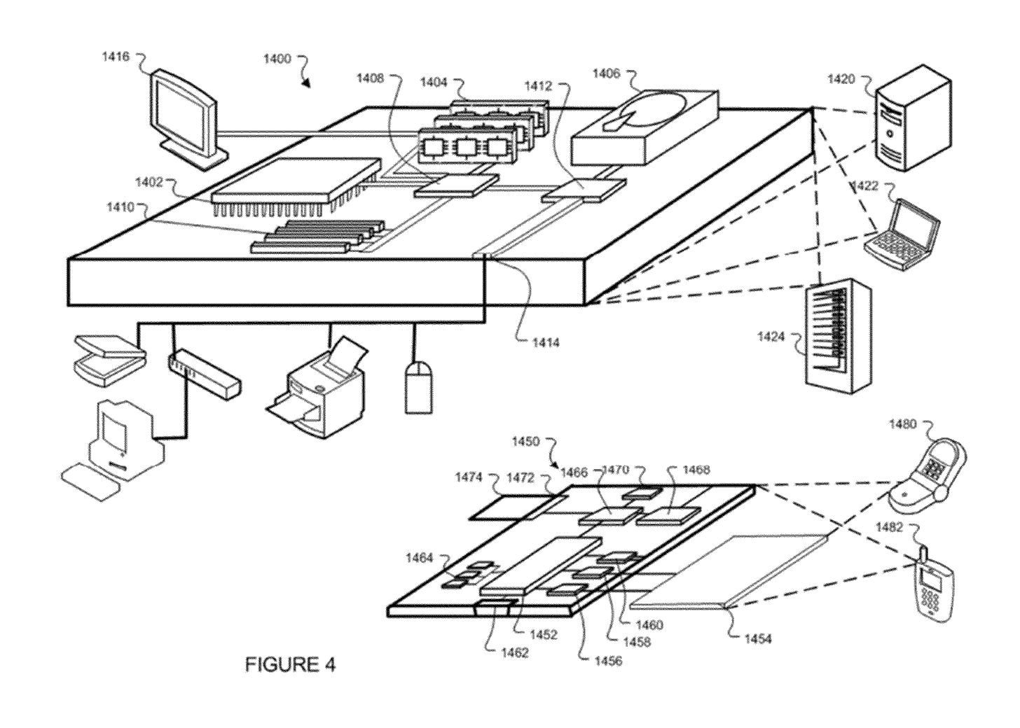

FIG. 4 is a diagram of an example of a computer device 1400 and a mobile computer device 1450, which may be used with the techniques described herein. Computing device 1400 is intended to represent various forms of digital computers, such as laptops, desktops, workstations, personal digital assistants, servers, blade servers, mainframes, and other appropriate computers. Computing device 1450 is intended to represent various forms of mobile devices, such as personal digital assistants, cellular telephones, smart phones, and other similar computing devices. The components shown here, their connections and relationships, and their functions, are meant to be exemplary only, and are not meant to limit implementations of the inventions described and/or claimed in this document.

Computing device 1400 includes a processor 1402, memory 1404, a storage device 1406, a high-speed interface 1408 connecting to memory 1404 and high-speed expansion ports 1410, and a low speed interface 1415 connecting to low speed bus 1414 and storage device 1406. Each of the components 1402, 1404, 1406, 1408, 1410, and 1415, are interconnected using various busses, and may be mounted on a common motherboard or in other manners as appropriate. The processor 1402 can process instructions for execution within the computing device 1400, including instructions stored in the memory 1404 or on the storage device 1406 to display graphical information for a GUI on an external input/output device, such as display 1416 coupled to high speed interface 1408. In other implementations, multiple processors and/or multiple buses may be used, as appropriate, along with multiple memories and types of memory. Also, multiple computing devices 1400 may be connected, with each device providing portions of the necessary operations (e.g., as a server bank, a group of blade servers, or a multi-processor system).

The memory 1404 stores information within the computing device 1400. In one implementation, the memory 1404 is a volatile memory unit or units. In another implementation, the memory 1404 is a non-volatile memory unit or units. The memory 1404 may also be another form of computer-readable medium, such as a magnetic or optical disk.

The storage device 1406 is capable of providing mass storage for the computing device 1400. In one implementation, the storage device 1406 may be or contain a computer-readable medium, such as a floppy disk device, a hard disk device, an optical disk device, or a tape device, a flash memory or other similar solid state memory device, or an array of devices, including devices in a storage area network or other configurations. A computer program product can be tangibly embodied in an information carrier. The computer program product may also contain instructions that, when executed, perform one or more methods, such as those described herein. The information carrier is a computer- or machine-readable medium, such as the memory 1404, the storage device 1406, memory on processor 1402, or a propagated signal.

The high speed controller 1408 manages bandwidth-intensive operations for the computing device 1400, while the low speed controller 1415 manages lower bandwidth-intensive operations. Such allocation of functions is exemplary only. In one implementation, the high-speed controller 1408 is coupled to memory 1404, display 1416 (e.g., through a graphics processor or accelerator), and to high-speed expansion ports 1410, which may accept various expansion cards (not shown). In the implementation, low-speed controller 1415 is coupled to storage device 1406 and low-speed expansion port 1414. The low-speed expansion port, which may include various communication ports (e.g., USB, Bluetooth, Ethernet, or wireless Ethernet) may be coupled to one or more input/output devices, such as a keyboard, a pointing device, a scanner, an optical reader, a fluorescent signal detector, or a networking device such as a switch or router, e.g., through a network adapter.

The computing device 1400 may be implemented in a number of different forms, as shown in the figure. For example, it may be implemented as a standard server 1420, or multiple times in a group of such servers. It may also be implemented as part of a rack server system 1424. In addition, it may be implemented in a personal computer such as a laptop computer 1422. Alternatively, components from computing device 1400 may be combined with other components in a mobile device (not shown), such as device 1450. Each of such devices may contain one or more of computing device 1400, 1450, and an entire system may be made up of multiple computing devices 1400, 1450 communicating with each other.

Computing device 1450 includes a processor 1452, memory 1464, an input/output device such as a display 1454, a communication interface 1466, and a transceiver 1468, among other components (e.g., a scanner, an optical reader, a fluorescent signal detector). The device 1450 may also be provided with a storage device, such as a microdrive or other device, to provide additional storage. Each of the components 1450, 1452, 1464, 1454, 1466, and 1468, are interconnected using various buses, and several of the components may be mounted on a common motherboard or in other manners as appropriate.

The processor 1452 can execute instructions within the computing device 1450, including instructions stored in the memory 1464. The processor may be implemented as a chipset of chips that include separate and multiple analog and digital processors. The processor may provide, for example, for coordination of the other components of the device 1450, such as control of user interfaces, applications run by device 1450, and wireless communication by device 1450.

Processor 1452 may communicate with a user through control interface 1458 and display interface 1456 coupled to a display 1454. The display 1454 may be, for example, a TFT LCD (Thin-Film-Transistor Liquid Crystal Display) or an OLED (Organic Light Emitting Diode) display, or other appropriate display technology. The display interface 1456 may comprise appropriate circuitry for driving the display 1454 to present graphical and other information to a user. The control interface 1458 may receive commands from a user and convert them for submission to the processor 1452. In addition, an external interface 1462 may be provide in communication with processor 1452, so as to enable near area communication of device 1450 with other devices. External interface 1462 may provide, for example, for wired communication in some implementations, or for wireless communication in other implementations, and multiple interfaces may also be used.

The memory 1464 stores information within the computing device 1450. The memory 1464 can be implemented as one or more of a computer-readable medium or media, a volatile memory unit or units, or a non-volatile memory unit or units. Expansion memory 1474 may also be provided and connected to device 1450 through expansion interface 1472, which may include, for example, a SIMM (Single In Line Memory Module) card interface. Such expansion memory 1474 may provide extra storage space for device 1450, or may also store applications or other information for device 1450. For example, expansion memory 1474 may include instructions to carry out or supplement the processes described herein, and may include secure information also. Thus, for example, expansion memory 1474 may be provide as a security module for device 1450, and may be programmed with instructions that permit secure use of device 1450. In addition, secure applications may be provided via the SIMM cards, along with additional information, such as placing identifying information on the SIMM card in a non-hackable manner.

The memory may include, for example, flash memory and/or NVRAM memory, as discussed below. In one implementation, a computer program product is tangibly embodied in an information carrier. The computer program product contains instructions that, when executed, perform one or more methods, such as those described herein. The information carrier is a computer- or machine-readable medium, such as the memory 1464, expansion memory 1474, memory on processor 1452, or a propagated signal that may be received, for example, over transceiver 1468 or external interface 1462.

Device 1450 may communicate wirelessly through communication interface 1466, which may include digital signal processing circuitry where necessary. Communication interface 1466 may provide for communications under various modes or protocols, such as GSM voice calls, SMS, EMS, or MMS messaging, CDMA, TDMA, PDC, WCDMA, CDMA2000, or GPRS, among others. Such communication may occur, for example, through radio-frequency transceiver 1468. In addition, short-range communication may occur, such as using a Bluetooth, WiFi, or other such transceiver (not shown). In addition, GPS (Global Positioning System) receiver module 1470 may provide additional navigation- and location-related wireless data to device 1450, which may be used as appropriate by applications running on device 1450.

Device 1450 may also communicate audibly using audio codec 1460, which may receive spoken information from a user and convert it to usable digital information. Audio codec 1460 may likewise generate audible sound for a user, such as through a speaker, e.g., in a handset of device 1450. Such sound may include sound from voice telephone calls, may include recorded sound (e.g., voice messages, music files, etc.) and may also include sound generated by applications operating on device 1450.

The computing device 1450 may be implemented in a number of different forms, as shown in the figure. For example, it may be implemented as a cellular telephone 1480. It may also be implemented as part of a smartphone 1482, personal digital assistant, or other similar mobile device.

Various implementations of the systems and techniques described herein can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms “machine-readable medium” and “computer-readable medium” refer to any computer program product, apparatus and/or device (e.g., magnetic discs, optical disks, memory, and Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

To provide for interaction with a user, the systems and techniques described herein can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described herein can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described herein), or any combination of such back end, middleware, or front end components. The components of the system can be interconnected by any form or medium of digital data communication (e.g., a communication network). Examples of communication networks include a local area network (“LAN”), a wide area network (“WAN”), and the Internet.

The computing system can include clients and servers. A client and server are generally remote from each other and typically interact through a communication network. The relationship of client and server arises by virtue of computer programs running on the respective computers and having a client-server relationship to each other.

In some cases, a computing system provided herein can be configured to include one or more sample analyzers. A sample analyzer can be configured to produce a plurality of signals about genomic DNA of at least one pair of human chromosomes of a cancer cell. For example, a sample analyzer can produce signals that are capable of being interpreted in a manner that identifies the genotype of loci along a chromosome. In some cases, a sample analyzer can be configured to carry out one or more steps of a SNP array-based assay or sequencing-based assay and can be configured to produce and/or capture signals from such assays. In some cases, a computing system provided herein can be configured to include a computing device. In such cases, the computing device can be configured to receive signals from a sample analyzer. The computing device can include computer-executable instructions or a computer program (e.g., software) containing computer-executable instructions for carrying out one or more of the methods or steps described herein. In some cases, such computer-executable instructions can instruct a computing device to analyze signals from a sample analyzer, from another computing device, from a SNP array-based assay, or from a sequencing-based assay. The analysis of such signals can be carried out to determine genotypes, homozygosity or other chromosomal aberration s at certain loci, regions of CA, the number of CA Regions, to determine the size of CA Regions, to determine the number of CA Regions having a particular size or range of sizes, to determine, e.g., whether or not a sample is positive for an HRD signature or the number of Indicator CA Regions in at least one pair of human chromosomes, in order to determine a likelihood that a cancer patient will respond to a cancer treatment regimen comprising niraparib.

In some cases, a computing system provided herein can include computer-executable instructions or a computer program (e.g., software) containing computer-executable instructions for formatting an output providing an indication about the number of CA Regions, the size of CA Regions, the number of CA Regions having a particular size or range of sizes, whether or not a sample is positive for an HRD signature, or the number of Indicator CA Regions in at least one pair of human chromosomes, each to assess the likelihood that a cancer patient will respond to a cancer treatment regimen comprising niraparib. In some cases, a computing system provided herein can include computer-executable instructions or a computer program (e.g., software) containing computer-executable instructions for indicating desired cancer treatment regimen for a particular patient based at least in part on the presence or absence of an HRD signature or on the number of Indicator CA Regions.

In some cases, a computing system provided herein can include a pre-processing device configured to process a sample (e.g., cancer cells) such that a SNP array-based assay or sequencing-based assay can be performed. Examples of pre-processing devices include, without limitation, devices configured to enrich cell populations for cancer cells as opposed to non-cancer cells, devices configured to lyse cells and/or extract genomic nucleic acid, and devices configured to enrich a sample for particular genomic DNA fragments.

This document also provides kits for assessing samples (e.g., cancer cells) as described herein. For example, this document provides kits for predicting response to treatment comprising niraparib by assessing cancer cells for the presence of an HRD signature or to determine the number of Indicator CA Regions in at least one pair of human chromosomes. A kit provided herein can include either SNP probes (e.g., an array of SNP probes for carrying out a SNP array-based assay described herein) or primers (e.g., primers designed for sequencing SNP regions via a sequencing-based assay) in combination with a computer program product containing computer-executable instructions for carrying out one or more of the methods or steps described herein (e.g., computer-executable instructions for determining the number of Indicator CA Regions). In some cases, a kit provided herein can include at least 500, 1000, 10,000, 25,000, or 50,000 SNP probes capable of hybridizing to polymorphic regions of human genomic DNA. In some cases, a kit provided herein can include at least 500, 1000, 10,000, 25,000, or 50,000 primers capable of sequencing polymorphic regions of human genomic DNA. In some cases, a kit provided herein can include one or more other ingredients for performing a SNP array-based assay or a sequencing-based assay. Examples of such other ingredients include, without limitation, buffers, sequencing nucleotides, enzymes (e.g., polymerases), etc. This document also provides the use of any appropriate number of the materials provided herein in the manufacture of a kit for carrying out one or more of the methods or steps described herein. For example, this document provides the use of a collection of SNP probes (e.g., a collection of 10,000 to 100,000 SNP probes) and a computer program product provided herein in the manufacture of a kit for assessing cancer cells for the presence of an HRD signature. As another example, this document provides the use of a collection of primers (e.g., a collection of 10,000 to 100,000 primers for sequencing SNP regions) and a computer program product provided herein in the manufacture of a kit for assessing cancer cells for the presence of an HRD signature.

The invention further provides methods of modifying treatment as described herein. A reference standard for treatment can be determined (“reference standard” or “reference level”) in order to direct treatment decisions. The reference standard may additionally comprise cutoff values or any other statistical attribute of the control population, such as a standard deviation from the mean HRD scores. In some embodiments, the control population may comprise healthy individuals or individuals with triple negative breast cancer.

In some embodiments, a patient is treated more or less aggressively than a reference therapy. A reference therapy is any therapy that is the standard of care for cancer. The standard of care can vary temporally and geographically, and a skilled person can easily determine the appropriate standard of care by consulting the relevant medical literature.

In some embodiments, based on a determination that an HRD score is a) greater than, b) less than, c) equal to, d) greater than or equal to, or e) less than or equal to a reference standard, treatment will be either 1) more aggressive, or 2) less aggressive than a standard therapy.

In some embodiments, a more aggressive therapy than the standard therapy comprises beginning treatment earlier than in the standard therapy. In some embodiments, a more aggressive therapy than the standard therapy comprises administering additional treatments than in the standard therapy. In some embodiments, a more aggressive therapy than the standard therapy comprises treating on an accelerated schedule compared to the standard therapy. In some embodiments, a more aggressive therapy than the standard therapy comprises administering additional treatments not called for in the standard therapy.

In some embodiments, a less aggressive therapy than the standard therapy comprises delaying treatment relative to the standard therapy. In some embodiments, a less aggressive therapy than the standard therapy comprises administering less treatment than in the standard therapy. In some embodiments, a less aggressive therapy than the standard therapy comprises administering treatment on a decelerated schedule compared to the standard therapy. In some embodiments, a less aggressive therapy than the standard therapy comprises administering no treatment.

In an embodiment, the practitioner adjusts the therapy (adds or excludes niraparib) based on a comparison between a reference HRD score and an HRD score from a patient. In one embodiment, the practitioner adjusts the therapy by selecting and administering a different combination of drugs comprising or not comprising niraparib. In one embodiment, the practitioner adjusts the therapy by adjusting niraparib dosage. In one embodiment, the practitioner adjusts the therapy by adjusting niraparib dose schedule. In one embodiment, the practitioner adjusts the therapy by adjusting length of therapy. In one embodiment, the practitioner adjusts the therapy by selecting and administering a different niraparib combination and adjusting drug dosage. In one embodiment, the practitioner adjusts the therapy by selecting and administering a different drug niraparib combination and adjusting dose schedule. In one embodiment, the practitioner adjusts the therapy by selecting and administering a different drug combination and adjusting length of therapy. In one embodiment, the practitioner adjusts the therapy by adjusting drug dosage and dose schedule. In one embodiment, the practitioner adjusts the therapy by adjusting drug dosage and adjusting length of therapy. In one embodiment, the practitioner adjusts the therapy by adjusting dose schedule and adjusting length of therapy. In one embodiment, the practitioner adjusts the therapy by selecting and administering a different drug, adjusting drug dosage, and adjusting dose schedule. In one embodiment, the practitioner adjusts the therapy by selecting and administering a different drug, adjusting drug dosage, and adjusting length of therapy. In one embodiment, the practitioner adjusts the therapy by selecting and administering a different drug, adjusting dose schedule, and adjusting length of therapy. In one embodiment, the practitioner adjusts the therapy by adjusting drug dosage, adjusting dose schedule, and adjusting length of therapy. In one embodiment, the practitioner adjusts the therapy by selecting and administering a different drug, adjusting drug dosage, adjusting dose schedule, and adjusting length of therapy.

In some embodiments, treatment comprises a less aggressive therapy than a reference therapy. In one embodiment a less aggressive therapy comprises not administering drugs and taking a “watchful waiting” approach. “Watchful-waiting,” also sometimes called “active surveillance,” also has its conventional meaning in the art. This generally means observation and regular monitoring without treatment of the underlying disease. Watching-waiting can also be suggested when the risks of surgery, radiation therapy, hormonal therapy, or chemotherapy, for example, outweighs the possible benefits. Other treatments can be started if symptoms develop, or if there are signs that the cancer growth is accelerating.

In one embodiment a less aggressive therapy comprises delaying treatment. In one embodiment a less aggressive therapy comprises selecting and administering a lower dose of niraparib. In one embodiment a less aggressive therapy comprises decreasing the frequency treatment. In one embodiment a less aggressive therapy comprises shortening length of therapy. In one embodiment, less aggressive therapy comprises selecting and administering less potent drugs and decreasing drug dosage. In one embodiment, less aggressive therapy comprises selecting and administering less potent drugs and decelerating dose schedule. In one embodiment, less aggressive therapy comprises selecting and administering less potent drugs and shortening length of therapy. In one embodiment, less aggressive therapy comprises decreasing drug dosage and decelerating dose schedule. In one embodiment, less aggressive therapy comprises decreasing drug dosage and shortening length of therapy. In one embodiment, less aggressive therapy comprises decelerating dose schedule and shortening length of therapy. In one embodiment, less aggressive therapy comprises selecting and administering less potent drugs, decreasing drug dosage, and decelerating dose schedule. In one embodiment, less aggressive therapy comprises selecting and administering less potent drugs, decreasing drug dosage, and shortening length of therapy. In one embodiment, less aggressive therapy comprises selecting and administering less potent drugs, decelerating dose schedule, and shortening length of therapy. In one embodiment, less aggressive therapy comprises decreasing drug dosage, decelerating dose schedule, and shortening length of therapy. In one embodiment, less aggressive therapy comprises selecting and administering less potent drugs, decreasing drug dosage, decelerating dose schedule, and shortening length of therapy. In some embodiments, a less aggressive therapy comprises administering only non-drug-based therapies.

In another aspect of the present application, treatment comprises a more aggressive therapy than a reference therapy. In one embodiment a more aggressive therapy comprises increased length of therapy. In one embodiment a more aggressive therapy comprises increased frequency of the dose schedule. In one embodiment, more aggressive therapy comprises selecting and administering more potent drugs and increasing drug dosage. In one embodiment, more aggressive therapy comprises selecting and administering more potent drugs and accelerating dose schedule. In one embodiment, more aggressive therapy comprises selecting and administering more potent drugs and increasing length of therapy. In one embodiment, more aggressive therapy comprises increasing drug dosage and accelerating dose schedule. In one embodiment, more aggressive therapy comprises increasing drug dosage and increasing length of therapy. In one embodiment, more aggressive therapy comprises accelerating dose schedule and increasing length of therapy. In one embodiment, more aggressive therapy comprises selecting and administering more potent drugs, increasing drug dosage, and accelerating dose schedule. In one embodiment, more aggressive therapy comprises selecting and administering more potent drugs, increasing drug dosage, and increasing length of therapy. In one embodiment, more aggressive therapy comprises selecting and administering more potent drugs, accelerating dose schedule, and increasing length of therapy. In one embodiment, more aggressive therapy comprises increasing drug dosage, accelerating dose schedule, and increasing length of therapy. In one embodiment, more aggressive therapy comprises selecting and administering more potent drugs, increasing drug dosage, accelerating dose schedule, and increasing length of therapy. In some embodiments, a more aggressive therapy comprises administering a combination of drug-based and non-drug-based therapies.

The invention will be further described in the following examples, which do not limit the scope of the invention described in the claims.

### EXAMPLES

### Example 1

### High HRD Threshold Value (e.g., One Example of an HRD Signature)

HRD scores were first determined by examining (1) associations between LOH, TAI, and LST and an HRD-combined score, (2) associations of clinical variables with an HRD-combined score, and (3) associations of clinical variables and the HRD-combined score with BRCA1/2 deficiency. This aspect of HRD score determination is disclosed, for example, in Example 4 of WO/2014/165785.

This example next demonstrates determination of high HRD. A threshold reference value was selected to have a high sensitivity for detecting HRD in breast and ovarian tumors that was nonspecific to treatment response or outcome. The total number of LOH, TAI, and LST Regions were determined. To calculate HRD scores, SNP data was analyzed using an algorithm that determines the most likely allele specific copy number at each SNP location. HRD-LOH was calculated by counting the number of LOH regions >15 Mb in length, but shorter than the length of a complete chromosome. HRD-TAI score was calculated by counting the number of regions >11 Mb in length with allelic imbalance that extend to one of the subtelomeres, but do not cross the centromere. HRD-LST score was the number of break points between regions longer than 10 Mb after filtering out regions shorter than 3 Mb. The combined score (HRD score) was the summation of the LOH/TAI/LST scores.

The training set was assembled from 4 different cohorts (497 breast and 561 ovarian cases). The set consisted of 78 breast and 190 ovarian tumors that were lacking a functional copy of BRCA1 or BRCA2, because the distribution of HRD scores in BRCA-deficient samples represents the distribution of scores in HRD samples in general. The threshold was set at the 5^{th} percentile of the HRD scores in the training set, and gives >95% sensitivity to detect HR deficiency. High HRD (or an HRD signature) was defined as having a reference score ≧42 (FIG. 14).

### Example 2

### HRD Predicts Niraparib Response in Triple Negative Breast Cancer

This example demonstrates how HRD scores as described herein can predict the efficacy of agents targeting HR deficiency in triple negative breast cancer (TNBC) samples. Analysis of a neoadjuvant TNBC cohort treated with niraparib was examined relative to the relationship between all three HRD scores and response. HRD measures genomic instability within tumor cells. As described in Example 1, HRD deficiency is defined as an HRD score ≧42. This example shows that niraparib significantly inhibited the growth of 6 TNBC patient-derived xenograft (PDX) models among 20 tested. All six sensitive models had HRD scores higher than 42.

### Methods

Mice were assigned to homogeneous groups of 10 animals when tumors reached a size of 70-200 mm³, and were treated with niraparib at 50 mg/kg or 75 mg/kg once a day for 28 days. The compound was delivered by gavage (p.o) under a volume of 125 ml for a 25-g mouse. Tumor volume was evaluated by measuring biweekly tumor diameters with a caliper.

For the HRD assay, DNA extracted from formalin fixed paraffin-embedded breast tumor tissues was used to create high complexity libraries that were hybridized to a custom Agilent SureSelect capture array carrying probes for 54,091 single nucleotide polymorphism sites distributed across the human genome, and 685 probes for BRCA1 and BRCA2 exons, exon boundaries, and promoter regions. The captured and enriched DNA was sequenced on an IIlumina HiSeq 2500 sequencer. Sequence reads were aligned to targets and assembled. Variant and large rearrangement detection was performed on sequence data from BRCA1 and BRCA2. Mutation identification was based on previously described criteria (Richards et al., Genet. Med. 10(4):294-300 (2008)). Sequence covering SNP positions was used to generate allelic imbalance profiles. A hidden Markov model (HMM) was used to define regions and breakpoints with these profiles. Allele specific copy number (ASCN) for each of the regions was determined using an algorithm similar to that previously described (Timms et al., Association of BRCA1/2 defects with genomic scores predictive of DNA damage repair deficiency among breast cancer subtypes, Breast Cancer Res, in press). TAI (number of regions of allelic imbalance that extend to one of the subtelomeres but do not cross the centromere) and LST (number of break points between regions longer than 10 Mb after filtering out regions shorter than 3 Mb) scores were calculated using the allelic imbalance profiles, while LOH (Number of subchromosomal LOH regions longer than 15 Mb) was calculated using ASCN.

### Results

This example shows that niraparib significantly inhibited the growth of 6 TNBC PDX models among 20 tests. Table 1 provides the HRD statuses of the basal breast cancer (BBC) PDX models.

TABLE 1
HRD status of BBC PDX models

| ID | HRD_Score | Mutation | BRCA1_LOH | BRCA2_LOH | BRCA1_Meth. |
|---|---|---|---|---|---|
| HBCx_12B | 78 | Wild-type | YES | NO | 14.0588 |
| HBCx_11 | 73 | BRCA1 c.1961del | YES | NO | 1.4563 |
| HBCx_6 | 72 | Wild-type | NO | NO | 34.1525 |
| HBCx_17 | 69 | BRCA2 c.6033_603del | NO | YES | 0.3052 |
| HBCx_15 | 68 | Wild-type | YES | NO | 33.8883 |
| HBCx_8 | 65 | BRCA1 c.241C > T | YES | NO | 0.3421 |
| T330 | 65 | BRCA1 c.3839_3844 delins5 | YES | NO | 0.0017 |
| HBCx_28 | 63 | BRCA1 c.212 + 3A > G | YES | NO | 0.9216 |
| HBCx_10 | 57 | BRCA2 c.9106C > T | NO | YES | 0.542 |
| HBCx_16 | 57 | Wild-type | YES | NO | 12.0913 |
| HBCx_9 | 55 | Wild-type | YES | NO | 13.8442 |
| HBCx_13B | 54 | Wild-type | NO | NO | 2.2643 |
| HBCx_14 | 53 | Wild-type | NO | NO | 0.492 |
| HBCx_1 | 50 | Wild-type | NO | NO | 9.399 |
| HBCx_5 | 38 | Wild-type | NO | NO | 0.4294 |
| HBCx_2# | 36 | Wild-type | NO | NO | 1.0793 |
| HBCx_33 | 32 | Wild-type | NO | NO | 8.3333 |
| HBCx_3 | 24 | Wild-type | NO | NO | 0.3999 |
| HBCx_19 | 17 | Wild-type | NO | NO | 0.8353 |
| HBCx_7 | 5 | Wild-type | NO | NO | 0.3702 |

#HBCx_2 contains BRCA2 mutations of uncertain significance;
LOH: loss of heterozygosity;
BRCA1_methylation: BRCA1 promoter methylation, >10% defined as methylated

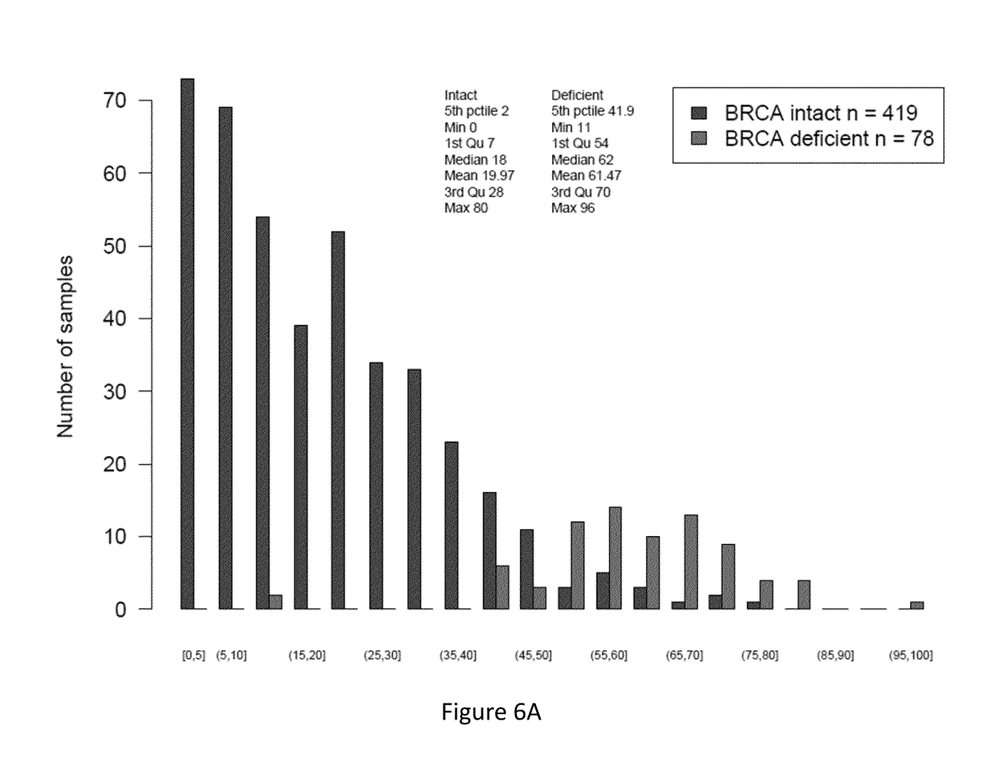

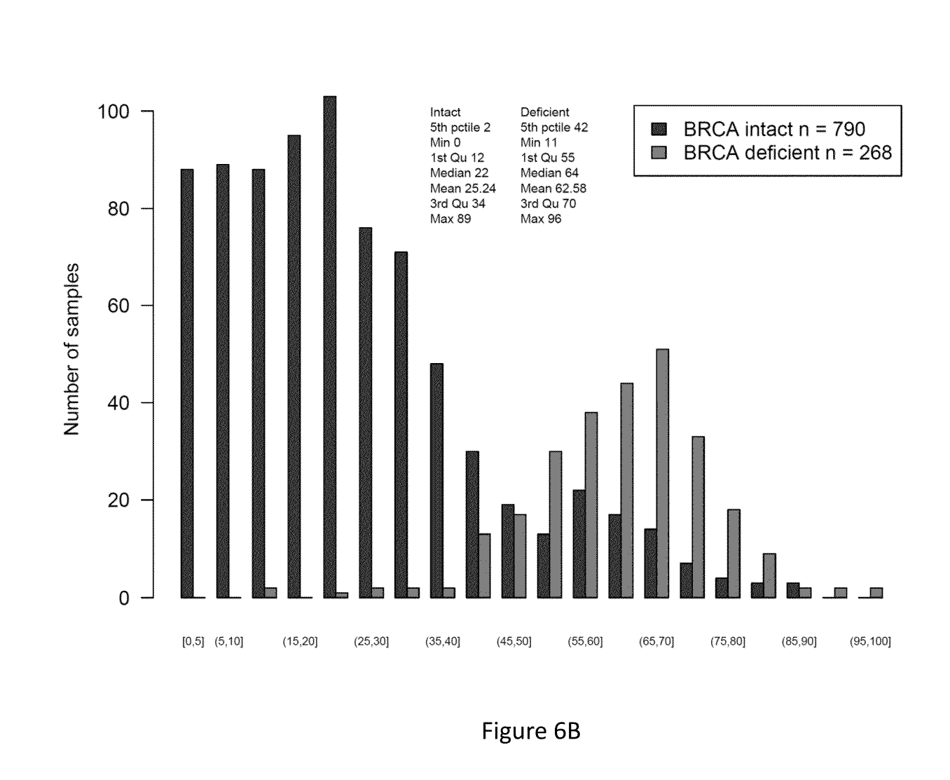

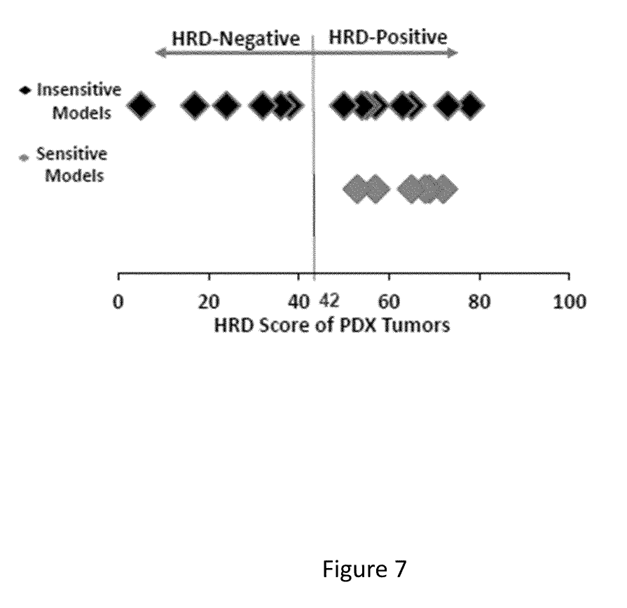

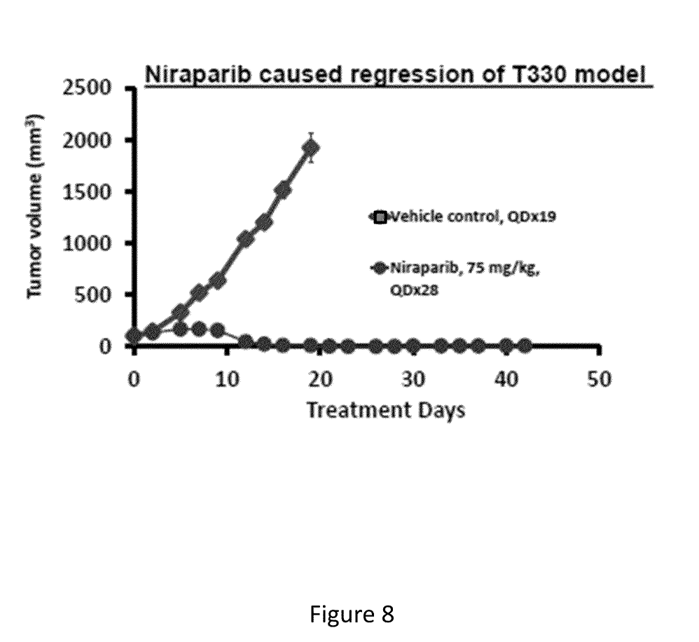

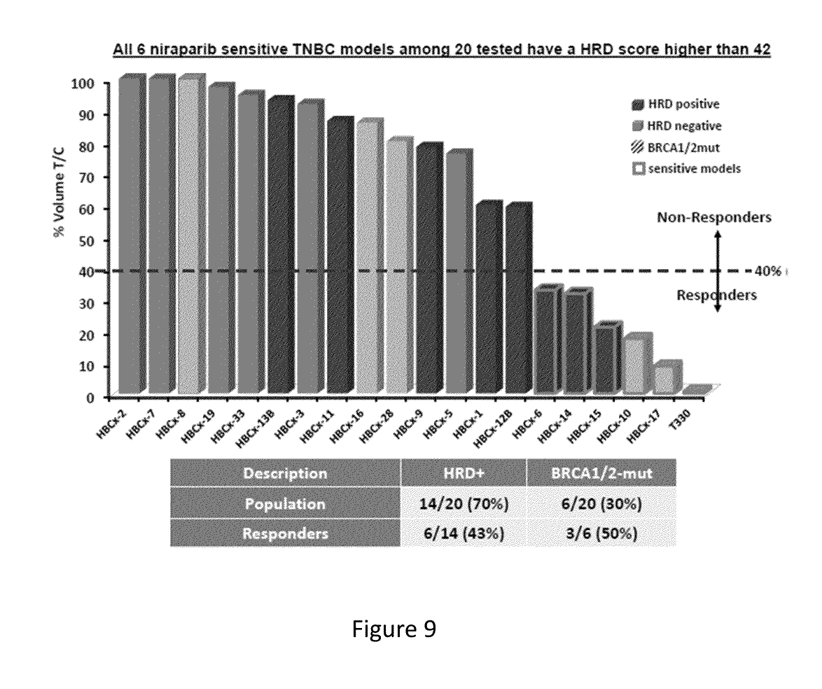

FIG. 6 illustrates the distribution of HRD scores in the training set of tumors. Among the 6 niraparib sensitive TNBC models, each were HRD positive with an HRD score greater than 42 (FIG. 7). FIG. 8 shows that niraparib caused regression of tumor volume in a T330 model. FIG. 9 shows that all 6 niraparib sensitive TNBC models were HRD positive, but reduced tumor volume.

In conclusion, this example demonstrates that 1) niraparib has robust activity in a subset of TNBC PDX models, 2) all niraparib sensitive TNBC models have an HRD score higher than 42, the cut-off defined as homologous recombination deficient, and 3) an HRD assay as described herein predicts niraparib sensitivity.

### Example 3

### HRD Predicts Niraparib Response in Ovarian Cancer

This example builds on Example 2 by demonstrating how HRD scores as described herein can predict efficacy of niraparib in ovarian cancer. A series of ovarian tumors were implanted orthotopically in mice and the resulting patient-derived xenograft (PDX) models were evaluated for niraparib sensitivity by ultrasound imaging. The BRCA status (mutated “BRCAmut” or wild-type “BRCAwt”) of each PDX model was evaluated, with BRCA1/2 variants identified by the test were classified in accordance with the recommendations of the American College of Medical Genetics and Genomics (ACMG) for standards in the interpretation and reporting of sequence variations. HRD scores were also determined for the PDX models as described in Examples 1 & 2.

TABLE 2

| Definition | Response rate |
|---|---|
| All models | 31% (8/26) |
| BRCA1/2 deficient | 50% (4/8) |
| HRD+ BRCA wt | 50% (4/8) |

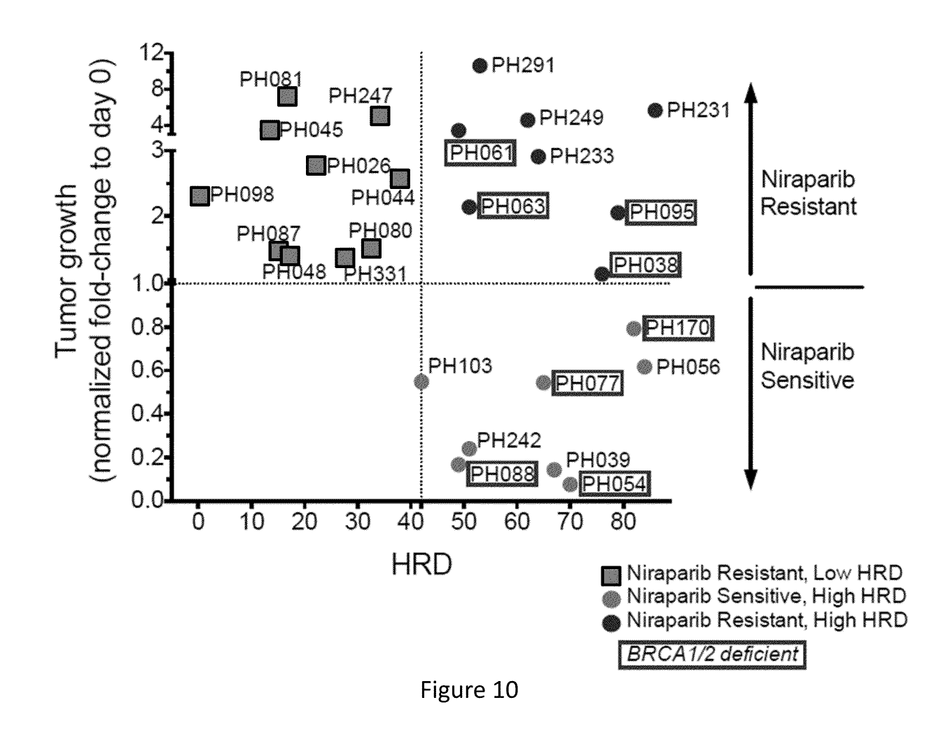

All sensitive models had an HRD score of ≧42. The response rate in BRCAmut was the same as in HRD+(BRCA wild-type or “BRCAwt”). The response for HRD deficient models (defined as either BRCAmut or HRD positive) was higher than in the overall cohort of PDX models (see FIGS. 10 & 11).

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An in vitro method of predicting patient response to a cancer treatment regimen comprising niraparib, the method comprising:
(1) determining in a patient sample the number of Indicator CA Regions comprising at least two types chosen from Indicator LOH Regions, Indicator TAI Regions, or Indicator LST Regions in at least two pairs of human chromosomes of a breast cancer cell; and
(2) diagnosing a patient in whose sample said number of Indicator LOH Regions, Indicator TAI Regions, and/or Indicator LST Regions is greater than a reference number as having an increased likelihood of responding to said cancer treatment regimen.

2. The method of claim 1, wherein said at least two pair of human chromosomes is representative of the entire genome.

3. The method of claim 1, wherein said Indicator CA Regions are determined in at least ten pairs of human chromosomes.

4. The method of claim 1 wherein the Indicator CA Regions analyzed in said patient sample comprise Indicator LOH Regions, Indicator TAI Regions, and Indicator LST Regions.

5. The method of claim 4 wherein a Combined CA Region Score is calculated from the number of Indicator CA Regions and in step (2) a patient in whose sample said Combined CA Region Score is greater than a reference Combined CA Region Score is diagnosed as having an increased likelihood of responding to said cancer treatment regimen.

6. The method of claim 1 wherein the reference number is 5 or greater.

7. The method of claim 6 wherein the reference number is 42.

8. The method of claim 5 wherein the reference Combined CA Region Scores is 5 or greater.

9. The method of claim 8, wherein the reference Combined CA Region Score is 42.

10. The method of claim 1, further comprising administering said cancer treatment regimen to said patient diagnosed as having an increased likelihood of responding to said cancer treatment regimen.

11. The method of claim 1, wherein the step of determining comprises hybridizing nucleic acids from the sample with probes.

12. An in vitro method of predicting patient response to a cancer treatment regimen comprising niraparib, the method comprising:
(1) determining in a patient sample the number of Indicator CA Regions comprising at least two types chosen from Indicator LOH Regions, Indicator TAI Regions, or Indicator LST Regions in at least two pairs of human chromosomes of a breast cancer cell;
(2) providing a test value derived from the number of said Indicator CA Regions;
(3) comparing said test value to one or more reference values derived from the number of said Indicator CA Regions in a reference population; and
(4) diagnosing a patient in whose sample said test value is greater than said one or more reference numbers as having an increased likelihood of responding to said cancer treatment regimen.

13. The method of claim 12, wherein said at least two pair of human chromosomes is representative of the entire genome.

14. The method of claim 12, wherein said Indicator CA Regions are determined in at least ten pairs of human chromosomes.

15. The method of claim 14, wherein the Indicator CA Regions analyzed in said patient sample comprise Indicator LOH Regions, Indicator TAI Regions, and Indicator LST Regions.

16. The method of claim 15, wherein a Combined CA Region Score is calculated from the number of Indicator CA Regions and in step (2) a patient in whose sample said Combined CA Region Score is greater than a reference Combined CA Region Score is diagnosed as having an increased likelihood of responding to said cancer treatment regimen.

17. The method of claim 12 wherein the reference number is 5 or greater.

18. The method of claim 17 wherein the reference number is 42.

19. The method of claim 16 wherein the reference Combined CA Region Scores is 5, 6, 7, 8, 9, 10, 11, 12, 12, 1/1, 15, 16, 18, 19, 20, 22, 2/1, 26, 28, 20, 22, 34-36-33-40-42-44-46-48-50 or greater.

20. The method of claim 19, wherein the reference Combined CA Region Score is 42.

21. The method of claim 12, further comprising diagnosing a patient in whose sample said test value is not greater than said one or more reference numbers as not having an increased likelihood of responding to said cancer treatment regimen and either (5)(a) recommending, prescribing, initiating or continuing a treatment regimen comprising niraparib in said patient diagnosed as having an increased likelihood of responding to said cancer treatment regimen; or (5)(b) recommending, prescribing, initiating or continuing a treatment regimen not comprising niraparib in said patient diagnosed as not having an increased likelihood of responding to said cancer treatment regimen.

22. The method of claim 12, comprising diagnosing a patient in whose sample said test value is at least 25% greater than said one or more reference numbers as having an increased likelihood of responding to said cancer treatment regimen.

23. The method of claim 12, wherein the determining comprises hybridizing nucleic acids from the sample with probes.

24. A method of treating cancer patients, comprising:
(1) determining in a patient sample the number of Indicator CA Regions comprising Indicator LOH Regions, Indicator TAI Regions, and Indicator LST Regions in at least two pairs of human chromosomes of a breast cancer cell;
(2) providing a test value derived from the number of said Indicator CA Regions;
(3) comparing said test value to one or more reference values derived from the number of said Indicator CA Regions in a reference population; and either
(4)(a) recommending, prescribing, initiating or continuing a treatment regimen comprising niraparib in a patient in whose sample the test value is greater than at least one said reference value; or
(4)(b) recommending, prescribing, initiating or continuing a treatment regimen comprising niraparib in a patient in whose sample the test value is not greater than at least one said reference value.

25-34. (canceled)

35. A method of analyzing a tumor sample, the method comprising the steps of:
(1) providing a tumor sample from a subject suffering from cancer, whose potential response to a cancer treatment regimen comprising niraparib is to be determined, wherein the tumor sample comprises nucleic acid;
(2) determining a value for Indicator CA Regions in the nucleic acid, which value incorporates numbers of at least two types of Indicator Regions selected from the group consisting of Indicator LOH Regions, Indicator TAI Regions, Indicator LST Regions, and combinations thereof, for at least two pairs of human chromosomes from cells in the tumor sample; and
(3) comparing the determined value with that of a corresponding reference value from a comparable tumor sample or samples from tumor(s) with known responsiveness to niraparib.

36-45 (canceled)
